(19) United States
(12) Patent Application Publication
(10) Pub. No.: US 2005/0034191 A1
(43) Pub. Date: Feb. 10, 2005

(54) Salt tolerant oil crops

(76) Inventor: Eduardo Blumwald, Davis, CA (US)

Correspondence Address: MORRISON & FOERSTER LLP, 425 MARKET STREET, SAN FRANCISCO, CA, 94105-2482, US

(21) Appl. No.: 10/617,624

(22) Filed: Jul. 10, 2003

Related U.S. Application Data
(60) Provisional application No. 60/395,656, filed on Jul. 12, 2002.

(51) Int. Cl. A01H 1/00; C12N 15/82; A01H 5/00

(52) U.S. Cl. 800/281; 800/306

(57) ABSTRACT

The subject invention includes salt tolerant, transgenic oil crop plants that produce normal or near normal distributions of fatty acids. The present invention also includes methods of making such transgenic oil crop plants. One preferred method is generating a transgenic oil crop plant that has an ectopically expressed NHX-1 related gene product.

### RELATED APPLICATIONS

This application claims the benefit of U.S. Provisional Application No. 60/395,656, filed Jul. 12, 2002, which is incorporated by reference herein in its entirety.

### FIELD OF THE INVENTION

This invention is in the field of agricultural biotechnology. In particular, this invention relates to salt tolerant, transgenic oil crop plants that produce normal or near normal distributions of fatty acids.

### BACKGROUND OF THE INVENTION

Agricultural productivity is severely affected by soil salinity, and the damaging effects of salt accumulation in agricultural soils have influenced ancient and modern civilizations. The detrimental effects of salt on plants are a consequence of both a water deficit that results from the relatively high solute concentrations in the soil, and a Na+-specific stress resulting from altered K+/Na+ ratios and Na+ ion concentrations that are inimical to plants. The alteration of ion ratios in the plant is due to the influx of Na+through pathways that function in the acquisition of K+. (Maathius, et al. (1999)) Full citations for the references cited herein are provided at the end of the Examples.

Salt tolerance is not exclusively associated with cellular Na⁺ homeostasis, but also involves adaptations to secondary effects of salinity such as oxidative damage and changes in the levels and composition of fatty acids of the major glycerolipids in roots and leaves of a wide range of plants. (Rais, et al. (1993)) Oil crops can be particularly affected by growth under elevated salt conditions. Changes in the level of fatty acid saturation/unsaturation have been reported as a response to salt stress, and a reduction in the levels of triacylglycerols containing unsaturated fatty acids has been reported in seed oil from cotton under salt stress. (Wu, et al. (1998); Yu, et al. (1998) and Smaoui, et al. (2000)) In addition, salt stress has been reported as leading to lowered levels of triolein, the major triacylglycerol in olive oil, in olives.

There is thus a tremendous need to be able to produce a salt tolerant oil crops that produce oils with normal or near normal distributions of fatty acids.

### SUMMARY OF THE INVENTION

In order to meet these needs, the present invention is directed to transgenic oil crops that are able to grow and produce oil in the presence of elevated salt concentrations. In particular, we show that transgenic Brassica napus plants overexpressing a vacuolar Na⁺/H⁺ antiport were able to grow, flower and produce seeds in the presence of 200 mM NaCl. Brassica napus, commonly known as canola or rapeseed, represents one of the most important oilseed crops that is being cultivated worldwide. The sustained growth of the transgenic plants, the seed yields and the quality of the seed oil demonstrate the potential use of these transgenic plants for agricultural use in saline soils.

One aspect of the present invention is directed to a non-naturally occurring non-halophyte oil crop plant comprising seeds with normal or near normal fatty acid distribution when cultivated in high salt. In some variations, the normal near normal fatty acid distribution is within 3%, within 5%, within 8%, within 10%, within 15%, or within 20% of the distribution in the corresponding plant variety grown in low to moderate salt. In another variation, the high salt is at least two times, at least three times, at least four times, at least five times, at least ten times, or at least twenty times the optimal salt concentration for the naturally occurring non-halophyte plant. In other variations, the plant may be canola, safflower, palm, coconut, cotton, flax, jojoba, peanut, castor, sesame, sunflower or soybean.

In another aspect of the present invention, the plant comprises a transgene. In still another variation, the transgene induces vacuolar accumulation of salt, secretion of the salt out of the cytoplasm or exclusion of salt from the cytoplasm. In one variation, the transgene comprises a first nucleic acid sequence encoding a Na+/H+ transporter or a plant derived Na+/H+ transporter. In another variation, the transgene comprises a first nucleic acid selected from the following group: a nucleic acid molecule of the coding strand shown in SEQ ID NO:1, or a complement thereof; a nucleic acid molecule encoding the amino acid sequence shown in SEQ ID NO:2; a nucleic acid molecule that hybridizes to the sequence set forth in SEQ ID NO:1 or the complement of the sequence set forth in SEQ ID NO:1 under highly stringent conditions that include at least one wash in 0.1×SSC, 0.1% SDS, at 65° C. for thirty minutes; and a nucleic acid molecule encoding a plant NHX transporter polypeptide that hybridizes to the sequence set forth in SEQ ID NO:1 or the complement of the sequence set forth in SEQ ID NO:1 under moderately stringent conditions that includes at least one wash in 0.1×SSC, 0.1% SDS, at 50° C. for thirty minutes. In still another variation, the transgene further comprises a promoter sequence operably linked to the first nucleic acid sequence. In yet another variation, the promoter is a constitutive promoter or an inducible promoter. In certain variations, the promoter may be selected from the group consisting of the 35 S promoter and the CaMV promoter.

An additional aspect of the present invention is a seed produced from any of the foregoing plants and variations thereof.

The present invention also includes methods of generating the foregoing. One variation includes transfecting a plant with a transcriptional regulatory element and identifying plants comprising seeds with normal or near normal fatty acid distribution when cultivated in high salt. In another variation, plants are transfected with a transcriptional regulatory element and identifying a plant wherein said transcriptional regulatory element has integrated operably linked to a Na+/H+ transporter. In yet another variation, the transcriptional regulatory element is a promoter, an enhancer element, a repressor element or a boundary element. In one variation, plants are transfected with a transgene comprising a Na+/H+ transporter and a plant comprising seeds with normal or near normal fatty acid distribution when cultivated in high salt is identified. In one variation, the Na+/H+ transporter gene is selected from the group consisting of a nucleic acid molecule of the coding strand shown in SEQ ID NO:1, or a complement thereof; a nucleic acid molecule encoding the amino acid sequence shown in SEQ ID NO:2; a nucleic acid molecule that hybridizes to the sequence set forth in SEQ ID NO:1 or the complement of the sequence set forth in SEQ ID NO:1 under highly stringent conditions that include at least one wash in 0.1×SSC, 0.1% SDS, at 65° C. for thirty minutes; and a nucleic acid molecule encoding a plant NHX transporter polypeptide that hybridizes to the sequence set forth in SEQ ID NO:1 or the complement of the sequence set forth in SEQ ID NO:1 under moderately stringent conditions that includes at least one wash in 0.1×SSC, 0.1% SDS, at 50° C. for thirty minutes.

### BRIEF DESCRIPTION OF THE DRAWINGS

FIG. 1 shows salt tolerance of wild-type plants and transgenic Brassica plants overexpressing AtNHX1 grown in the presence of 200 mM NaCl. Wild-type (wt) and homozygous plants showing high (X1OE₁), medium (X1OE₂) and low (X1OE₃) levels of expression were grown in the presence of 200 mM NaCl. Plants shown after 10 weeks of growth. Inset: Western blots of leaf tonoplast-enriched membrane fractions isolated from wild-type and transgenic plants with low, medium and high levels of expression of AtNHX1. Blots were probed with antibodies raised against the C-terminus of AtNHX1. Equal amounts of protein (20 μg) were loaded in each lane. Relative molecular masses are indicated on the left.

FIG. 2 shows Na⁺ and K⁺ contents of leaves and roots from wild-type plants grown at 10 mM NaCl (black bars) and transgenic plants (X1OE1) grown at 10 mM NaCl (white bars) and 200 mM NaCl (hatched line bars). (A) Na⁺ content; (B) K⁺ content. Leaves and roots were collected from fifteen plants from each treatment, the material pooled in three groups and ion contents measured as described in Materials and Methods. Values are the Mean±S.D (n=3).

FIG. 3 shows proline, soluble sugars, protein and total nitrogen contents of leaves and roots from wild-type plants grown at 10 mM NaCl (black bars); and transgenic plants (X1OE₁) grown at 10 mM NaCl (white bars) and 200 mM NaCl (hatched line bars). (A) Proline content; (B) soluble sugar content; (C) total protein content; (D) total nitrogen content. Leaves and roots were collected from fifteen plants from each treatment, the material pooled in three groups and contents measured as described in Materials and Methods. Values are the Mean±S.D (n=3).

FIG. 4 shows fatty acid composition of the minor chloroplastic lipids from wild-type plants grown at 10 mM NaCl (black bars); and transgenic plants grown (X1OE₁) at 10 mM NaCl (white bars) and 200 mM NaCl (hatched line bars). (A) Sulfoquinovosyldiacylglycerol; (B) Phosphatidylglycerol. Leaves were collected as leaf discs from 15 plants from each treatment, the material pooled in to 3 groups of 2 g each and contents purified and measured as described in Material and Methods. Values are the Mean±S.D (n=5).

FIG. 5 shows fatty acid composition of seeds from wild-type plants grown in 10 mM NaCl (black bars) and transgenic plants (X1OE₁) grown in the presence of 200 mM NaCl (hatched line bars). Seeds were collected from individual plants and batches of 3 seeds per plant were used for each measurement. Values are the Mean±S.D (n=5).

### BRIEF DESCRIPTION OF THE TABLES

Table I shows a comparison of the yield of a non-naturally occurring salt tolerant oil crop in the presence of 10 mM NaCl and 200 mM NaCl and the yield of the naturally occurring oil crop of the same variety grown in the presence of 10 mM NaCl.

Table II shows the a comparison of the lipid content of leaves and roots of a non-naturally occurring salt tolerant oil crop grown in the presence of 10 mM and 200 mM NaCl and a naturally occurring oil crop of the same variety grown in the presence of 10 mM NaCl.

Table III shows a representative list of NXH related gene products.

Table IV shows the salinity levels that lead to a 25% relative decrease in yield and a 50% relative decrease in yield for various crop plants, including soybean, an oil crop plant.

### DETAILED DESCRIPTION OF THE INVENTION

Oil Crops of the Present Invention

The present invention provides a non-naturally occurring oil crop that is characterized by increased salt tolerance and a normal or near normal distribution of seed oils when grown under elevated salt conditions. A preferred method of making such crop plants is to ectopically express a nucleic acid molecule encoding an NHX related gene product. The NHX related gene product can have, for example, substantially the amino acid sequence of an NHX ortholog such as those described in Table III.

As defined herein, an “oil crop” is any crop plant capable of producing oil. Such crop plants include rape/canola; coconut; cotton; flax; palm; olive; jojoba; peanut; castor; safflower; sesame; sunflower and soybean.

In one embodiment, the invention provides a transgenic oil crop characterized by increased salt tolerance due to ectopic expression of an exogenous nucleic acid molecule encoding an NHX-related gene product. The nucleic acid molecule encoding the NHX-related gene product can be operatively linked to an exogenous regulatory element such as a constitutive regulatory element or crop-selective regulatory element.

The present invention is directed to the surprising discovery that the NHX-1 increases salt tolerance in oil crops and results in a normal or near normal distribution of fatty acids in seed oils. As disclosed herein, transgenic Brassica plants overexpressing AtNHX1 were able to grow, flower and produce seeds in the presence of 200 mM NaCl. The seeds produced in the presence of 200 mM NaCl had a normal or near normal distribution of fatty acids when compared to seeds produced in the presence of 10 mM NaCl.

As further disclosed herein, overexpression of AtNHX1 in Brassica plants results in increased salt tolerance as compared to the salt tolerance of wild type Brassica plants. As set forth in The Example, constitutive expression of NHX1 under control of a 35 S promoter resulted in oil crops having increased salt tolerance as compared to the salt tolerance of wild type plants. In view of the presence and expression of the NHX ortholog, as detailed in Table III, the skilled artisan will recognize that an NHX-related gene product, such as an ortholog of NHX-1, can be used in the methods of the present invention, for example, to produce transgenic plants having the characteristics disclosed herein. Thus, the invention provides a non-naturally occurring oil crop such as a transgenic Brassica plant, characterized by increased salt tolerance due to ectopic expression of a nucleic acid molecule encoding an NHX-1 related gene product.

As used herein, the term “non-naturally occurring,” when used in reference to an oil crop, means an oil crop that has been genetically modified by man. A transgenic oil crop of the invention, for example, is a non-naturally occurring plant that contains an exogenous nucleic acid molecule, such as a nucleic acid molecule encoding an NHX-related gene product and, therefore, has been genetically modified by human intervention. In addition, an oil crop that contains, for example, a mutation in an endogenous NHX-related gene product regulatory element or coding sequence as a result of calculated exposure to a mutagenic agent, such as a chemical mutagen, or an “insertional mutagen,” such as a transposon, also is considered a non-naturally occurring oil crop, since it has been genetically modified by human intervention. Furthermore, a plant generated by cross breeding different strains and varieties are also considered a “non-naturally occurring plant,” because the selection and breeding is performed by human intervention. In contrast, an oil crop containing only spontaneous or naturally occurring mutations is not a “non-naturally occurring oil crop” as defined herein and, therefore, is not encompassed within the invention. One skilled in the art understands that, while a non-naturally occurring oil crop typically has a nucleotide sequence that is altered as compared to a similar naturally occurring oil crop, a non-naturally occurring oil crop also can be genetically modified by human intervention without altering its nucleotide sequence, for example, by modifying its methylation pattern.

The term “ectopically,” as used herein in reference to expression of a nucleic acid molecule, refers to an expression pattern in a non-naturally occurring plant that is distinct from the expression pattern in a comparable naturally occurring plant. Thus, one skilled in the art understands that ectopic expression of a nucleic acid molecule encoding an NHX-related gene product can refer to expression in a cell type other than a cell type in which the nucleic acid molecule normally is expressed, or at a time other than a time at which the nucleic acid molecule normally is expressed, or at a level other than the level at which the nucleic acid molecule normally is expressed. For example, under control of a constitutive promoter such as the cauliflower mosaic virus 35S promoter, NHX is expressed is expressed at higher than normal levels in oil crops and, thus, is ectopically expressed.

The term “increased salt tolerance,” as used herein in reference to a non-naturally occurring oil crop variety of the invention, means a significantly increased salt tolerance as compared to the salt tolerance of a corresponding oil crop variety lacking a genetic modification introduced by human intervention such as an ectopically expressed nucleic acid molecule encoding an NHX-related gene product such as a wild type oil crop. As disclosed herein in The Example, transgenic Brassica napus plants ectopically expressing NHX-1 have an increased salt tolerance as compared to wild type Brassica plants.

It is recognized that there can be natural variation in the salt tolerance of a particular plant species or variety. However, the salt tolerance of an oil crop using a method of the invention readily can be identified by sampling a population of the oil crop and determining that the normal distribution of salt tolerance is higher, on average, than the normal distribution of an oil crop lacking an ectopically expressed nucleic acid molecule encoding an NHX-related gene product. Thus, production of non-naturally occurring oil crops of the invention provides a means to skew the normal distribution of salt tolerance of a plant, such that the salt tolerance is, on average, at least about 5% greater, 10% greater, 20% greater, 30% greater, 50% greater, 75% greater, 100% greater, 200% greater, 300% greater, 400% greater or 500% greater than in the corresponding plant species that does not contain an ectopically expressed nucleic acid molecule encoding an NHX-related gene product.

The term “normal or near normal distribution of fatty acids,” as used herein in reference to a non-naturally occurring oil crop of the invention, means a distribution of major fatty acids (greater than 5% of the oil) in the oil produced from the non-naturally occurring oil crop grown in high salt wherein the fraction of each major fatty acid is nearly normal when compared to the distribution of major fatty acids in the oil produced from the same non-naturally occurring oil crop grown in moderate salt. As disclosed herein in The Example, transgenic Brassica napus plants ectopically expressing NHX-1 have a normal or near normal distribution of fatty acids when grown in the presence of 200 mM NaCl as compared to the transgenic plants grown at 10 mM NaCl. The term, however, does not include the distribution of fatty acids in the leaves or the roots.

It is recognized that there can be natural variation in the distribution of fatty acids in oils produced by a particular plant species or variety. However, the distribution of fatty acids in oils produced by an oil crop using a method of the invention readily can be identified by sampling a population of the oil crop and determining that the normal distribution of fatty acids is nearly normal when the oil crop is cultivated under high salt, on average, when compared to the distribution of fatty acids when the oil crop is grown in moderate to low salt. Thus, production of non-naturally occurring oil crops of the invention provides a means to produce plants which when grown in high salt produce oils with a distribution of major fatty acids in the oil produced wherein the fraction of each major fatty acid is within 3%, within 5%, within 8%, within 10%, within 15%, or within 20% of the fraction of the same major fatty acid in oil produced by the corresponding plant grown in low to moderate salt.

The term “non-halophyte,” as used herein means a plant that is not naturally morphologically and/or physiologically adapted to grow in salt rich soils or salt laden air. A non-halophyte is a plant variety that has a relative yield decrease of 50% or more at 200 mM NaCl (the equivalent of about 20 dS/m) when compared to the plant variety grown at optimal salinity levels which are below 200 mM NaCl. The invention is suitable for even more salt sensitive plant varieties which have a relative yield decrease of 50% or more at 180 mM NaCl, 160 mM NaCl, 140 mM NaCl, 120 mM NaCl, 100 mM NaCl or 80 mM NaCl. Table IV lists the relative yield decrease for various non-halophyte crop plants.

The term “elevated salinity conditions” as used herein refers to the salinity level at which a plant variety has a relative yield decrease of 50% when compared to the plant variety grown at lower optimal salinity levels.

The term “saline-intolerant plants” as used herein means a plant variety that cannot complete its life cycle in growth media containing a salinity level above 200 mM NaCl. The invention is suitable for even more highly saline-intolerant plant varieties that cannot complete their life cycle in growth media containing a salinity level above 180 mM NaCl, 160 mM NaCl, 140 mM NaCl, 120 mM NaCl, 100 nM NaCl and even 7 mM NaCl.

Methods of Making the Oil Crop Plants

The following methods are illustrative of some of the methods that may be used to make the oil crops of the present invention. With the Example herein, one of skill in the art will now recognize that many methods may be used to generate the oil crops of the present invention based upon dealing with salt accumulation in the cytosol. Examples include without limitation: replacement of salt sensitive enzymes in the fatty acid biosynthetic pathway with salt tolerant enzymes; exclusion of salt from the cytosol; secretion of salt out of the cytosol; and compartmentalization of the salt away from the cytosol such as in the vacuole. A preferred method is generating an oil crop ectopically expressing an NHX-related gene product.

As used herein, the term “NHX-related gene product” means a gene product that has the same or similar function as At NHX such that, when ectopically expressed in an oil crop, normal salt tolerance is altered such that oil crops with increased salt tolerance are produced. Arabidopsis NHX-1 is an example of an NHX-related gene product as defined herein.

An NHX-related gene product generally is characterized, in part, as containing a putative action binding domain and an amiloride binding domain. An NHX-related gene product also generally is characterized by having an amino acid sequence that has at least about 40% amino acid identity with the amino acid sequence of Arabidopsis NHX-1. An NHX-related gene product can have, for example, an amino acid sequence with greater than about 45% amino acid sequence identity with Arabidopsis NHX-1, preferably greater than about 50% amino acid identity with Arabidopsis NHX-1, preferably greater than about 55% amino acid sequence identity with Arabidopsis NHX-1, preferably greater than about 60% amino acid identity with Arabidopsis NHX-1, preferably greater than about 65% amino acid sequence identity with Arabidopsis NHX-1, preferably greater than about 75% amino acid identity with Arabidopsis NHX-1, more preferably greater than about 85% amino acid identity with Arabidopsis NHX-1, and can be a sequence having greater than about 90%, 95% or 97% amino acid identity with Arabidopsis NHX-1.

Preferably, an NHX-related gene product is orthologous to the plant species in which it is ectopically expressed. A nucleic acid molecule encoding Brassica NHX, for example, can be ectopically expressed in a Brassica plant to produce a non-naturally occurring Brassica variety characterized by an increased salt tolerance and normal or near-normal distributions of fatty acids. Similarly, a nucleic acid molecule encoding oil plant NHX, for example, can be ectopically expressed in an oil crop to produce a non-naturally occurring oil crop characterized by producing salt tolerant oil crops.

A nucleic acid molecule encoding an NHX-related gene product also can be ectopically expressed in a heterologous plant to produce a non-naturally occurring plant characterized by an increased salt tolerance. NHX proteins have been cloned from a number of plant species (including Arabidopsis, tomato, sugar beets, petunia, rice, etc) indicating that they are widely conserved throughout the plant species. NHX-related gene products such as NHX orthologs also can be conserved and can function across species boundaries to result in an increased salt tolerance. Thus, ectopic expression of a nucleic acid molecule encoding NHX in a heterologous plant can alter the salt tolerance of the plant. Furthermore, a nucleic acid molecule encoding NHX, for example, can be ectopically expressed in more distantly related heterologous plants, including oil crops, and, upon ectopic expression, can alter salt tolerance.

As used herein, the term “NHX-related gene product” encompasses an active segment of an NHX-related gene product, which is a polypeptide portion of an NHX-related gene product that, when ectopically expressed, increases salt tolerance. An active segment can be, for example, an amino terminal, internal or carboxy terminal fragment of NHX-1 that, when ectopically expressed in an oil crop, results in an increased salt tolerance. The skilled artisan will recognize that a nucleic acid molecule encoding an active segment of an NHX-related gene product can be used to generate a plant of the invention characterized by an increased salt tolerance and in the related methods and kits of the invention described further below.

An active segment of an NHX-related gene product can be identified using the methods described in The Example or using other routine methodology. Briefly, an oil crop such as Brassica napus can be transformed with a nucleic acid molecule under control of a constitutive regulatory element such as a tandem CaMV 35S promoter. Biochemical analysis of the plant and plant growth observations reveals whether an oil crop ectopically expressing a particular polypeptide portion has an increased salt tolerance. For analysis of a large number of polypeptide portions of an NHX-related gene product, nucleic acid molecules encoding the polypeptide portions can be assayed in pools, and active pools subsequently subdivided to identify the active nucleic acid molecule.

In one embodiment, the invention provides a non-naturally occurring oil crop that is characterized by an increased salt tolerance due to ectopic expression of a nucleic acid molecule encoding an NHX-related gene product having substantially the amino acid sequence of an NHX ortholog. As used herein, the term “NHX ortholog” means an ortholog of Arabidopsis NHX-1 and refers to an NHX-related gene product that, in a particular plant variety, has the highest percentage homology at the amino acid level to Arabidopsis NHX-1. An NHX-1 ortholog can be, for example the NHX-1 orthologs described in Table III. Novel NHX ortholog cDNAs can be isolated from additional plant species using a nucleotide sequence as a probe and methods well known in the art of molecular biology (Glick and Thompson (eds.), Methods in Plant Molecular Biology and Biotechnology, Boca Raton, Fla.: CRC Press (1993); Sambrook et al. (eds.), Molecular Cloning: A Laboratory Manual (Second Edition), Plainview, N.Y.: Cold Spring Harbor Laboratory Press (1989), each of which is incorporated herein by reference).

As used herein, the term “substantially the amino acid sequence,” when used in reference to an NHX ortholog, is intended to mean a polypeptide or polypeptide segment having an identical amino acid sequence, or a polypeptide or polypeptide segment having a similar, non-identical sequence that is considered by those skilled in the art to be a functionally equivalent amino acid sequence. For example, an NHX-related gene product having substantially the amino acid sequence of Arabidopsis NHX-1 can have an amino acid sequence identical to the sequence of Arabidopsis NHX-1, or a similar, non-identical sequence that is functionally equivalent. In particular, a gene product that has “substantially the amino acid sequence” of an NHX ortholog can have one or more modifications such as amino acid additions, deletions or substitutions, including conservative or non-conservation substitutions, relative to the NHX-1 amino acid sequence, for example, provided that the modified polypeptide retains substantially the ability to increase salt tolerance when the nucleic acid molecule is ectopically expressed in the plant. Comparison of sequences for substantial similarity can be performed between two sequences of any length and usually is performed with sequences between about 6 and 1200 residues, preferably between about 10 and 100 residues and more preferably between about 25 and 35 residues. Such comparisons for substantial similarity are performed using methodology routine in the art.

The preferred percentage of sequence similarity for sequences of NHX orthologs includes nucleotide sequences having at least about: 48% similarity to SEQ ID NO:1. The similarity may also be at least about: 60% similarity, 75% similarity, 80% similarity, 90% similarity, 95% similarity, 97% similarity, 98% similarity, 99% similarity, or more preferably at least about 99.5% similarity, wherein the polypeptide has Na+/H+ transporter activity. The invention also includes salt tolerant oil crop plants made by transgenic expression of nucleic acid molecules encoding polypeptides, with the polypeptides having at least about: at least about: 48% similarity to SEQ iID NO:2. The similarity may also be at least about: 60% similarity, 75% similarity, 80% similarity, 90% similarity, 95% similarity, 97% similarity, 98% similarity, 99% similarity, or more preferably at least about 99.5% similarity, wherein the polypeptide Na+/H+ has transporter activity, to SEQ ID NO:2 (or a partial sequence thereof) considering conservative amino acid changes, wherein the polypeptide has Na+/H+ transporter activity. Sequence similarity is preferably calculated as the number of similar amino acids in a pairwise alignment expressed as a percentage of the shorter of the two sequences in the alignment. The pairwise alignment is preferably constructed using the Clustal W program, using the following parameter settings: fixed gap penalty=10, floating gap penalty=10, protein weight matrix=BLOSUM62. Similar amino acids in a pairwise alignment are those pairs of amino acids which have positive alignment scores defined in the preferred protein weight matrix (BLOSUM62). The protein weight matrix BLOSUM62 is considered appropriate for the comparisons described here by those skilled in the art of bioinformatics. (The reference for the clustal w program (algorithm) is Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680; and the reference for BLOSUM62 scoring matrix is Henikoff, S. and Henikoff, J. G. (1993) Performance evaluation of amino acid substitution matrices. Proteins, 7:49-61.)

It is understood that minor modifications of primary amino acid sequence can result in an NHX-related gene product that has substantially equivalent or enhanced function as compared to the NHX ortholog from which it was derived. Further, various molecules can be attached to an NHX ortholog or active segment thereof, for example, other polypeptides, antigenic or other peptide tags, carbohydrates, lipids, or chemical moieties. Such modifications are included within the term NHX ortholog as defined herein.

One or more point mutations can be introduced into a nucleic acid molecule encoding an NHX ortholog to yield a modified nucleic acid molecule using, for example, site-directed mutagenesis (see Wu (Ed.), Meth. In Enzymol. Vol. 217, San Diego: Academic Press (1993); Higuchi, “Recombinant PCR” in Innis et al. (Ed.), PCR Protocols, San Diego: Academic Press, Inc. (1990), each of which is incorporated herein by reference). Such mutagenesis can be used to introduce a specific, desired amino acid insertion, deletion or substitution; alternatively, a nucleic acid sequence can be synthesized having random nucleotides at one or more predetermined positions to generate random amino acid substitutions. Scanning mutagenesis also can be useful in generating a modified nucleic acid molecule encoding substantially the amino acid sequence of an NHX ortholog.

Modified nucleic acid molecules can be routinely assayed for the ability to alter normal plant development such that salt tolerance is increased. For example, a nucleic acid molecule encoding substantially the amino acid sequence of an NHX ortholog can be ectopically expressed, for example, using a constitutive regulatory element such as the CaMV 35S promoter or using a tissue-specific regulatory element such as a seed-selective regulatory element as described further below. If such ectopic expression results in a seed plant in which seeds of increased size are produced, the modified polypeptide or segment is an “NHX ortholog” as defined herein.

Other functional equivalent forms of the NHX-related gene product encoding nucleic acids can be identified using conventional DNA-DNA or DNA-RNA hybridization techniques. These nucleic acid molecules and the AtNHX sequences can be modified without significantly affecting their activity.

The oil crops of the present invention may therefore also be made by generating transgenic plants containing nucleic acid molecules that hybridize to one SEQ ID NO:1 or their complementary sequences, and that encode expression for peptides or polypeptides exhibiting substantially equivalent activity as that of an AtNHX polypeptide produced by SEQ ID NO:1 or their variants. Such nucleic acid molecules preferably hybridize to the sequences under low, moderate (intermediate), or high stringency conditions. (See Sambrook et al. (Most recent edition) Molecular Cloning: A Laboratory Manual, Cold Spring Harbor Laboratory Press, Cold Spring Harbor, N.Y.).

As used herein, the phrase “low stringency hybridization conditions” refers the following conditions and equivalents thereto: hybridization at 5×SSC, 2% SDS, and 100 μg/ml single stranded DNA at 40° C. for 8 hours, followed by at least one wash in 2×SSC, 0.2% SDS, at 40° C. for thirty minutes.

As used herein, the phrase “moderate stringency hybridization conditions” refers the following conditions and equivalents thereto: hybridization at 5×SSC, 2% SDS, and 100 μg/ml single stranded DNA at 50° C. for 8 hours, followed by at least one wash in 0.1×SSC, 0.1% SDS, at 50° C. for thirty minutes.

As used herein, the phrase “high stringency hybridization conditions” refers the following conditions and equivalents thereto: hybridization at 5×SSC, 2% SDS, and 100 μg/ml single stranded DNA at 65° C. for 8 hours, followed by at least one wash in 0.1×SSC, 0.1% SDS, at 65° C. for thirty minutes.

The invention also provides a transgenic oil crop that is characterized by increased salt tolerance resulting from ectopic expression of an exogenous nucleic acid molecule encoding an NHX-related gene product. In a transgenic oil crop of the invention, the ectopically expressed exogenous nucleic acid molecule encoding an NHX-related gene product can be operatively linked to an exogenous regulatory element. In one embodiment, the invention provides a transgenic plant characterized by increased salt tolerance having an ectopically expressed exogenous nucleic acid molecule encoding an NHX-related gene product that is operatively linked to a constitutive regulatory element. The invention provides, for example, a transgenic oil crop that is characterized by an increased salt tolerance due to ectopic expression of an exogenous nucleic acid molecule encoding substantially the amino acid sequence of an NHX ortholog operatively linked to a cauliflower mosaic virus 35S promoter.

In another embodiment, an exogenous constitutive or inducible regulatory element may be introduced to the plant such that the exogenous regulatory element is operably linked to an endogenous gene and alters the expression pattern of the gene in a manner that provides salt tolerance that leads to normal or near normal distributions of fatty acids when the plant is grown in the presence of high salt. One example of this would be to transfect a plant with the cauliflower mosaic virus 35S promoter such that the promoter integrates in a way that it is operably linked to one of the plant's endogenous NHX-related genes.

In yet another embodiment, an exogenous NHX-related gene may be introduced to the plant such that the exogenous NHX-related gene is operably linked to an endogenous regulatory element which directs the expression of the gene in a manner that provides salt tolerance that leads to normal or near normal distributions of fatty acids when the plant is grown in the presence of high salt. One example of this would be to transfect a plant with the at NHX1 gene such that the gene integrates in a way that it is operably linked to one of the plant's endogenous strong promoters.

As used herein, the term “transgenic” refers to an oil crop that contains an exogenous nucleic acid molecule, which can be derived from the same plant species or from a heterologous plant species.

The term “exogenous,” as used herein in reference to a nucleic acid molecule and a transgenic plant, means a nucleic acid molecule originating from outside the plant. An exogenous nucleic acid molecule can have a naturally occurring or non-naturally occurring nucleotide sequence. One skilled in the art understands that an exogenous nucleic acid molecule can be a heterologous nucleic acid molecule derived from a different plant species than the plant into which the nucleic acid molecule is introduced or can be a nucleic acid molecule derived from the same plant species as the oil crop into which it is introduced.

The term “operatively linked,” as used in reference to a regulatory element and a nucleic acid molecule, such as a nucleic acid molecule encoding an NHX-related gene product, means that the regulatory element confers regulated expression upon the operatively linked nucleic acid molecule. Thus, the term “operatively linked,” as used in reference to an exogenous regulatory element such as a constitutive regulatory element and a nucleic acid molecule encoding an NHX-related gene product, means that the constitutive regulatory element is linked to the nucleic acid molecule encoding an NHX-related gene product such that the expression pattern of the constitutive regulatory element is conferred upon the nucleic acid molecule encoding the NHX-related gene product. It is recognized that a regulatory element and a nucleic acid molecule that are operatively linked have, at a minimum, all elements essential for transcription, including, for example, a TATA box.

As used herein, the term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant generally is widely expressed in a large number of cell and tissue types.

A variety of constitutive regulatory elements useful for ectopic expression in a transgenic plant of the invention are well known in the art. The cauliflower mosaic virus 35S (CaMV 35S) promoter, for example, is a well-characterized constitutive regulatory element that produces a high level of expression in all plant tissues (Odell et al., Nature 313:810-812 (1985)). The CaMV 35S promoter can be particularly useful due to its activity in numerous diverse plant species (Benfey and Chua, Science 250:959-966 (1990); Futterer et al., Physiol. Plant 79:154 (1990); Odell et al., supra, 1985). A tandem 35S promoter, in which the intrinsic promoter element has been duplicated, confers higher expression levels in comparison to the unmodified 35S promoter (Kay et al., Science 236:1299 (1987)). Other constitutive regulatory elements useful for ectopically expressing a nucleic acid molecule encoding an NHX-related gene product in a transgenic oil crop of the invention include, for example, the cauliflower mosaic virus 19S promoter; the Figwort mosaic virus promoter; and the nopaline synthase (nos) gene promoter (Singer et al., Plant Mol. Biol. 14:433 (1990); An, Plant Physiol. 81:86 (1986)).

Additional constitutive regulatory elements including those for efficient ectopic expression in monocots also are known in the art, for example, the pEmu promoter and promoters based on the rice Actin-1 5′ region (Last et al., Theor. Appl. Genet. 81:581 (1991); Mcelroy et al., Mol. Gen. Genet. 231:150 (1991); Mcelroy et al., Plant Cell 2:163 (1990)). Chimeric regulatory elements, which combine elements from different genes, also can be useful for ectopically expressing a nucleic acid molecule encoding an NHX-related gene product (Comai et al., Plant Mol. Biol. 15:373 (1990)). One skilled in the art understands that a particular constitutive regulatory element is chosen based, in part, on the plant species in which a nucleic acid molecule encoding an NHX-related gene product is to be ectopically expressed and on the desired level of expression.

An exogenous regulatory element useful in a transgenic oil crop of the invention also can be an inducible regulatory element, which is a regulatory element that confers conditional expression upon an operatively linked nucleic acid molecule, where expression of the operatively linked nucleic acid molecule is increased in the presence of a particular inducing agent or stimulus as compared to expression of the nucleic acid molecule in the absence of the inducing agent or stimulus. Particularly useful inducible regulatory elements include copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Roder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11: 1251-1259 (1992)).

An inducible regulatory element useful in the transgenic oil crops of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)). Additional inducible regulatory elements include salicylic acid inducible regulatory elements (Uknes et al., Plant Cell 5:159-169 (1993); Bi et al., Plant J. 8:235-245 (1995)); plant hormone-inducible regulatory elements (Yamaguchi-Shinozaki et al., Plant Mol. Biol. 15:905 (1990); Kares et al., Plant Mol. Biol. 15:225 (1990)); and human hormone-inducible regulatory elements such as the human glucocorticoid response element (Schena et al., Proc. Natl. Acad. Sci. USA 88:10421 (1991)).

It should be recognized that a non-naturally occurring oil crop of the invention, which contains an ectopically expressed nucleic acid molecule encoding an NHX-related gene product, also can contain one or more additional modifications, including naturally and non-naturally occurring mutations that can, for example, increase salt tolerance.

The invention further provides a method of producing a non-naturally occurring oil crop characterized by an increased salt tolerance. The method is practiced by ectopically expressing a nucleic acid molecule encoding an NHX-related gene product in the plant, whereby salt tolerance is increased due to ectopic expression of the nucleic acid molecule. In one embodiment, the method is practiced by introducing an exogenous nucleic acid molecule encoding an NHX-related gene product into the plant.

As discussed above, the term “ectopically” refers to expression of a nucleic acid molecule encoding an NHX-related gene product in a cell type other than a cell type in which the nucleic acid molecule is normally expressed, at a time other than a time at which the nucleic acid molecule is normally expressed or at an expression level other than the level at which the nucleic acid molecule normally is expressed.

Actual ectopic expression of an NHX-related gene product is dependent on various factors. The ectopic expression can be widespread expression throughout most or all plant tissues or can be expression restricted to a small number of plant tissues, and can be achieved by a variety of routine techniques. Mutagenesis, including seed or pollen mutagenesis, can be used to generate a non-naturally occurring oil crop, in which a nucleic acid molecule encoding an NHX-related gene product is ectopically expressed. Ethylmethane sulfonate (EMS) mutagenesis, transposon mediated mutagenesis or T-DNA mediated mutagenesis also can be useful in ectopically expressing an NHX-related gene product to produce a seed plant that produces seeds of increased size (see, generally, Glick and Thompson, supra, 1993). While not wishing to be bound by any particular mechanism, ectopic expression in a mutagenized plant can result from inactivation of one or more negative regulators of NHX, for example.

Ectopic expression of an NHX-related gene product also can be achieved by expression of a nucleic acid molecule encoding an NHX-related gene product from a heterologous regulatory element or from a modified variant of its own promoter. Heterologous regulatory elements include constitutive regulatory elements, which result in expression of the NHX-related gene product in a limited number of plant tissues.

Ectopic expression of a nucleic acid molecule encoding an NHX-related gene product can be achieved using an endogenous or exogenous nucleic acid molecule encoding an NHX-related gene product. A recombinant exogenous nucleic acid molecule can contain a heterologous regulatory element that is operatively linked to a nucleic acid sequence encoding an NHX-related gene product. Methods for producing the desired recombinant nucleic acid molecule under control of a heterologous regulatory element and for producing a non-naturally occurring plant of the invention are well known in the art (see, generally, Sambrook et al., supra, 1989; Glick and Thompson, supra, 1993).

An exogenous nucleic acid molecule can be introduced into a plant for ectopic expression using a variety of transformation methodologies including Agrobacterium-mediated transformation and direct gene transfer methods such as electroporation and microprojectile-mediated transformation (see, generally, Wang et al. (eds), Transformation of Plants and Soil Microorganisms, Cambridge, UK: University Press (1995), which is incorporated herein by reference). Transformation methods based upon the soil bacterium Agrobacterium tumefaciens are particularly useful for introducing an exogenous nucleic acid molecule into an oil crop. The wild type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or, preferably, binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art (Glick and Thompson, supra, 1993). Wounded cells within the plant tissue that have been infected by Agrobacterium can develop organs de novo when cultured under the appropriate conditions; the resulting transgenic shoots eventually give rise to transgenic plants that ectopically express a nucleic acid molecule encoding an NHX-related gene product. Agrobacterium also can be used for transformation of oil crops as described in Bechtold et al., C. R. Acad. Sci. Paris. Life Sci. 316:1194-1199 (1993) (which is incorporated herein by reference). Agrobacterium-mediated transformation is useful for producing a variety of transgenic oil crops (Wang et al., supra, 1995) including transgenic plants of the Brassicaceae family, such as rapeseed and flax.

Microprojectile-mediated transformation also can be used to produce a transgenic oil crop that ectopically expresses an NHX-related gene product. This method, first described by Klein et al. (Nature 327:70-73 (1987), which is incorporated herein by reference), relies on microprojectiles such as gold or tungsten that are coated with the desired nucleic acid molecule by precipitation with calcium chloride, spermidine or PEG. The microprojectile particles are accelerated at high speed into an angiosperm tissue using a device such as the BIOLISTIC PD-1000 (Biorad; Hercules Calif.).

Microprojectile-mediated delivery or “particle bombardment” is especially useful to transform oil crops that are difficult to transform or regenerate using other methods. Microprojectile-mediated transformation has been used, for example, to generate a variety of transgenic plant species, including cotton, tobacco, corn, hybrid poplar and papaya (see Glick and Thompson, supra, 1993) as well as cereal crops such as wheat, oat, barley, sorghum and rice (Duan et al., Nature Biotech. 14:494498 (1996); Shimamoto, Curr. Opin. Biotech. 5:158-162 (1994), each of which is incorporated herein by reference). In view of the above, the skilled artisan will recognize that Agrobacterium-mediated or microprojectile-mediated transformation, as disclosed herein, or other methods known in the art can be used to produce a transgenic oil crop of the invention.

If desired, a kit of the invention also can contain a plant expression vector. As used herein, the term “plant expression vector” means a self-replicating nucleic acid molecule that provides a means to transfer an exogenous nucleic acid molecule into an oil crop host cell and to express the molecule therein. Plant expression vectors encompass vectors suitable for Agrobacterium-mediated transformation, including binary and cointegrating vectors, as well as vectors for physical transformation.

Plant expression vectors can be used for transient expression of the exogenous nucleic acid molecule, or can integrate and stably express the exogenous sequence. One skilled in the art understands that a plant expression vector can contain all the functions needed for transfer and expression of an exogenous nucleic acid molecule; alternatively, one or more functions can be supplied in trans as in a binary vector system for Agrobacterium-mediated transformation.

In addition to containing a nucleic acid molecule encoding an NHX-related gene product operatively linked to a seed-selective regulatory element, a plant expression vector of the invention can contain, if desired, additional elements. A binary vector for Agrobacterium-mediated transformation contains one or both T-DNA border repeats and can also contain, for example, one or more of the following: a broad host range replicon, an ori T for efficient transfer from E. coli to Agrobacterium, a bacterial selectable marker such as ampicillin and a polylinker containing multiple cloning sites.

A plant expression vector for physical transformation can have, if desired, a plant selectable marker and can be based on a vector such as pBR322, pUC, pGEM and M13, which are commercially available, for example, from Pharmacia (Piscataway, N.J.) or Promega (Madison, Wis.). In plant expression vectors for physical transformation of an oil crop, the T-DNA borders or the ori T region can optionally be included but provide no advantage.

The invention will be better understood by reference to the following non-limiting example.

### EXAMPLE

Materials and Methods

Plant Material.

Seeds of Brassica napus cv. Westar were rinsed with running water for two days, surface-sterilized with a solution of 10% commercial bleach (0.525% sodium hypochlorite) and 0.1% SDS for 5 min and washed three times with sterile distilled water. Seeds were germinated on Murashige and Skoog medium (MS). Cotyledon explants were excised from 7 day-old seedlings. The binary Ti vector pBI121 was used for transformation. (Jefferson, et al. (1986)) The GUS gene of the binary vector was replaced with the AtNHX1 gene to gain the new expression construct pHZX1. The new construct was electroporated into Agrobacterium tumefaciens strain LBA4404. For co-cultivation, 1 ml of pHZX1 containing LBA4404 Agrobacterium was inoculated into 15 ml LB medium containing 50 mg.1-1 kanamycin, 50 mg.1-1 rifampicin and 200 μM acetone-syringone. The culture was incubated one day at room temperature under constant shaking (250 rpm) and then diluted one time with liquid MS medium. The cotyledon explants were submerged in the Agrobacterium solution for 3 min, blotted on sterile paper towels and returned to the feeder plates for 2 days of co-cultivation. After co-cultivation, the explants were transferred to a selective regeneration medium. (Moloney, et al. (1989)) Regenerated shoots were transferred to fresh medium bi-weekly. When the green shoots were 1-2 cm tall, they were separated from the calli and transferred onto rooting medium which contained modified MS medium supplemented with 3.7 mM KNO3, 4.1 mM NH4NO3, 0.5 mM MgSO4, 75 mg/l Kanamycin, 200 mg/l Ampicillin and 1 mg/l indole butyric acid. Under these conditions, about 98% shoots formed roots in two weeks. Rooted shoots were transplanted to soil, plants were grown and seeds (T1) collected. T1 seeds were grown on MS medium plates containing 15 mg/l kanamycin, plants were grown and homozygous seeds (T2) selected. For salt tolerance experiments, wild type and transgenic seeds (T2) overexpressing the vacuolar Na+/H+ antiport were germinated in 250 ml pots containing pro-mix BX peat moss, perlite and vermiculite medium (Premier Brands, New Rochelle, N.Y.) and grown in the greenhouse. Two weeks after germination the plants were watered bi-weekly with a nutrient solution with low (10 mM) or high (200 mM) concentrations of NaCl. Sixty of each wild-type and transgenic plants were distributed in two groups of thirty plants each, and each group was watered with a solution with low or high salinity. The nutrient solution was obtained by mixing 1.2 g per liter of stock fertilizer (6-11-31, Plant-Prod, Brampton, Ontario) and 1 g per liter of Ca(NO3)2. The final nutrient solution contained (in mM) 15 N, 2 P, 6.5 K, 4 Ca, 2 Mg, 9.5 S, micronutrients and was supplemented with 5 mM or 200 mM NaCl. Day temperature was maintained at 28±2° C. and night temperature was 20±2° C. Relative humidity was maintained at 50±10%. Plants were grown under a 14 h/10 h light/dark photoperiod. Supplemental lighting consisted of eight high-pressure sodium lamps, and resulted in a total flux (sunlight and supplemental light) of approximately 1,450 μmol m⁻²S⁻¹.

Membrane Isolation and Western Blots.

Tonoplast-enriched membrane fractions were isolated from leaves of 10-week-old plants as described. (Zhang, et al. (2001)) Western blots were performed as described.

Leaf, Root and Seed Chemical and Lipid Analysis.

Roots were rinsed with distilled water and leaves and roots were collected from fifteen plants from each treatment, pooled in three groups, dried at 70° C. for 24 h and the material was ground to a fine powder. Seeds were collected from the rest of the plants 3 weeks later. For the determination of soluble sugars and proline contents, 100 mg of each pool was resuspended in 2 ml of water, sonicated and centrifuged for 10 min at 2,500×g. Soluble sugar, proline and protein contents were determined in the supernatant as described. (Blumwald, et al. (1985);Dubois, et al. (1956) and Bates, et al. (1973)) Ion contents were determined by atomic absorption spectrophotometry. Lipids were extracted from 2 g of mature leaf tissue or 3 g of root tissue with chloroform/methanol (2;1,v/v) and purified as previously described. (Williams, et al. (1970)) Lipid classes were separated by thin-layer chromatography (TLC) on silica gel G plates containing ammonium sulfate using acetone/benzene/water (91:30:8,v/v). (Khan, et al. (1977)) The lipids were scraped from the plate and trans-esterified with 1 mL 1.5 M HCl in dry methanol in a microwave oven as previously described and the fatty acid methyl esters (FAME) were extracted from the methanolic HCl with hexane. (Khan, et al. (1993)) Seed oil fatty acid compositions were determined by direct trans-esterification of whole seeds using the microwave technique. The FAME were analyzed by gas-liquid chromatography using a Hewlett-Packard model 5890 gas-liquid chromatograph (Hewlett-Packard, Mississauga, Ontario, Canada) with a 30 m×0.25 mm ID DB-23 capillary column (J & W Scientific, Folsom, Calif.) programmed from 160° C. to 210° C. at 3° C. min-1. The FAME were estimated quantitatively using methylpentadecanoate as an internal standard.

Results

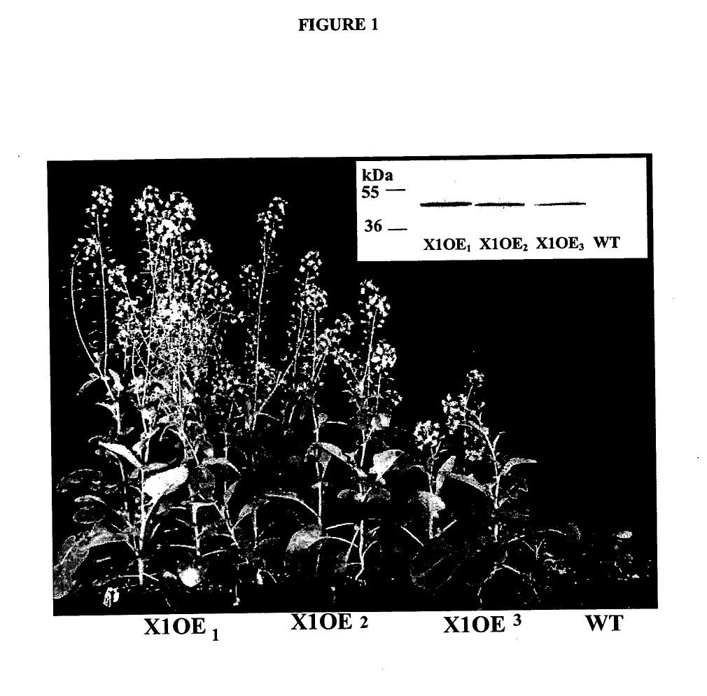

A construct containing the AtNHX1 was introduced into the genome of Brassica napus cv Westar. Sixty-four transgenic plants were obtained and nine homozygous lines from these transgenic plants were obtained in the T2 generation (data not shown). In order to assess whether the enhanced expression of the vacuolar Na⁺/H⁺ antiport would allow plants to grow in high salt conditions, wild-type and three lines of transgenic plants (with relatively low, medium and high levels of transgene expression) were grown in the presence of 200 mM NaCl (FIG. 1), a concentration that inhibits the growth of almost all crop plants. The overexpression of the vacuolar Na⁺/H⁺ antiport did not affect the growth of the transgenic plants since similar growth was observed when the wild-type and the transgenic plants were grown in the presence of 10 mM NaCl (Table I). The growth of the wild-type plants was severely affected by the presence of 200 mM NaCl in the growth solution, plant growth was inhibited and the plants were severely stunted (FIG. 1). On the other hand, the transgenic plants grew, flowered and produced seeds (FIG. 1, Table I). The growth of the transgenic plants in 200 mM NaCl was correlated with the increased levels of AtNHX1 protein (FIG. 1). Immunoblots of membrane fractions isolated from wild-type and transgenic plants only detected AtNHX1 in the tonoplast-enriched fractions from transgenic plants indicating the proper targeting of the Na⁺/H⁺ antiport to the tonoplast (FIG. 1).

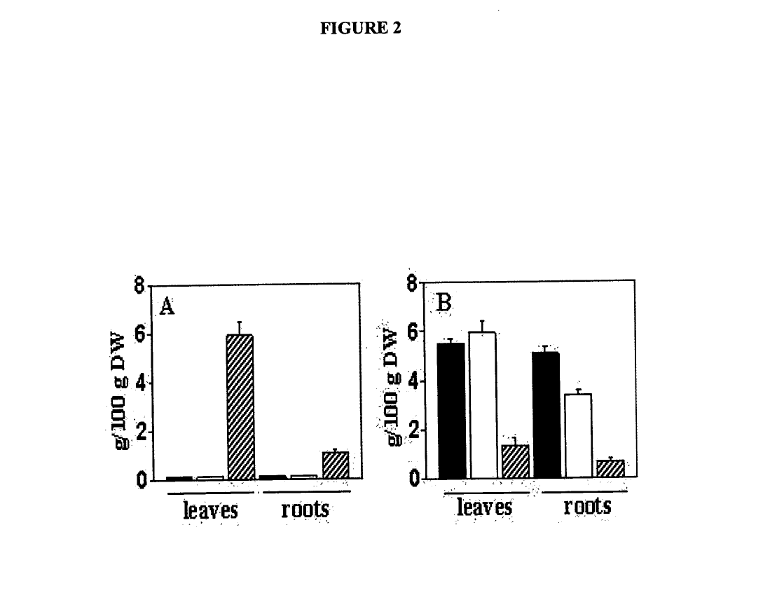

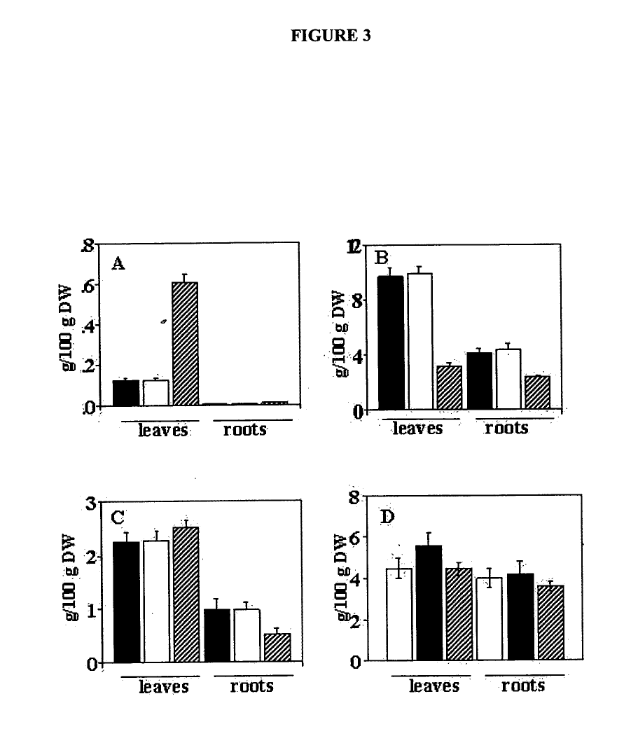

We determined the Na⁺, K⁺, soluble sugars, proline, total protein, nitrogen and phosphorus contents of wild-type and transgenic plants grown at low (10 mM) NaCl and transgenic plants grown at high (200 mM) NaCl (FIGS. 2 and 3). At low salinity, no significant differences were seen in the leaf and root Na⁺ content from wild-type and transgenic plants (FIG. 2). Dramatic changes were seen in transgenic plants grown at high salinity. A 70- and 9-fold increase in Na⁺ content was seen in the leaves and roots of these plants, respectively. The K⁺ content of leaves and roots of transgenic plants growing at high salinity decreased by 75% and 82%, respectively. While the leaf soluble sugars content declined during growth at high salinity (FIG. 3), a 6-fold increase in proline content was seen in high-salt grown leaves. There were no significant differences in N (FIG. 3) or total P content (data not shown). It should be noted that a comparison with wild-type plants grown at high salinity was not possible since all of the wild-type plants grown in these conditions were dead.

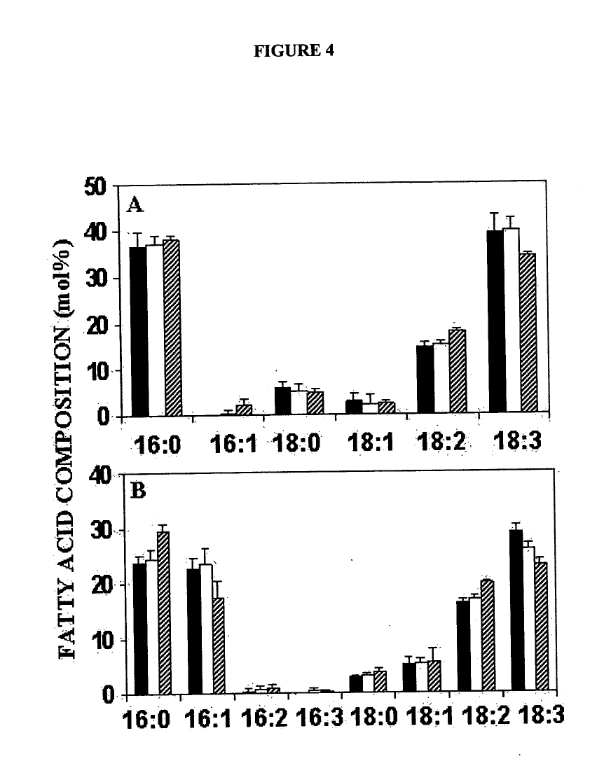

The major root and leaf lipids from wild-type grown at low salinity and transgenic plants grown at low and high salinity were analyzed (Table II). No significant differences in the major chloroplastic and extraplastidic lipids were found. The fatty acid composition of the two major extraplastidic lipids, phosphatidylcholine (PC) and phosphatidylethanolamine (PE) did not differ in either the 16/18 C ratio or the degree of unsaturation (not shown). Similarly, no differences were observed in the fatty acid compositions of the chloroplastic lipids digalactosyldiacylglycerol (DGDG) and mongalactosyldiacylglycerol (MGDG). Neither DGDG (synthesized predominantly through the eukaryotic pathway) nor MGDG (synthesized predominantly through the prokaryotic pathway) showed any significant difference in the 16/18 C ratio or degree of unsaturation (results not shown). Some differences, however, were seen in the minor chloroplastic lipids, sulfoquinovosyldiacylglycerol (SQDG) and phosphatidylglycerol (PG) (FIG. 4).

Although the 16/18 C ratios were the same, there were differences in the degree of unsaturation of the 18 C fatty acids in both SQDG and PG from transgenic plants grown in 200 mM NaCl. The ratio of palmitic acid (16:0)/trans-Δ³-hexadecenoic acid (trans16:1) in PG from transgenic plants grown in 200 mM NaCl was significantly higher than in plants grown in 10 mM NaCl.

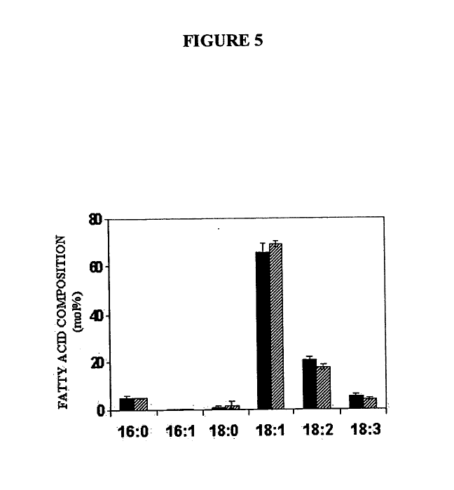

In roots, the predominant lipids are the extraplastidic phospholipids. Although the levels of MGDG, synthesized predominantly through the eukaryotic pathway in roots, are similar to those in leaves, the other plastidic lipids are found in very low quantities in roots. There were no significant differences in the fatty acid compositions of PC, PE and MGDG from wild type and transgenic plants grown at 10 mM NaCl or 200 mM NaCl (results not shown). Total fatty acid analyses of the seed oil did not differ significantly in seeds from wild-type plants grown in 10 mM NaCl and transgenic plants grown in 200 mM NaCl (FIG. 5). Quantitatively and qualitatively the seed oil from the transgenic plants is identical with seed oil from the wild-type plants.

Discussion

Taken together, our results demonstrate the ability of the transgenic oil crops to utilize salty water for growth. In spite of the high Na⁺ content in the leaves of the transgenic plants grown at 200 mM NaCl, these plants were able to grow, flower and set seed. These results clearly demonstrate that the enhanced accumulation of Na⁺, mediated by the vacuolar Na⁺/H⁺ antiport, allowed the transgenic plants to mitigate the toxic effects of Na⁺. (Apse, et al. (1999) and Zhang, et al. (2001)) Notably, transgenic plants grown at 200 mM NaCl produced numbers of seeds similar to those of wild-type plants grown at low salinity. Moreover, qualitative and quantitative analyses of the oil content showed no significant differences between seeds from wild-type grown at low salinity and transgenic plants grown at high salinity. It should be noted that although our experiments were carried out in the greenhouse, our results were obtained under growth conditions with a relatively low humidity and high light intensity. The leaf and root K⁺ contents of the transgenic plants grown in 200 mM NaCl were lower than those from plants grown in low salinity. Adaptation of plants to saline environments not only depends on their ability to ameliorate the toxic effects of Na⁺ per se, but also on their ability to overcome salt-induced impaired nutrient acquisition. (Marschner (1995)) This is of particular importance with regards to K⁺ uptake and K⁺ homeostasis. Potassium concentrations in plant cells are kept under homeostatic control with cytosolic K⁺ concentrations in the order of 100-200 mM. (Wyn Jones, et al. (1983)) When exposed to relatively low NaCl concentrations, Na⁺ ions can promote growth of many plants, in particular at low K⁺ concentrations in the growth medium. (Elzam, et al. (1969)) Under high salinity conditions, Na⁺ ions may displace K⁺ from its carrier binding sites and this competition results in impaired K⁺ uptake and lower K⁺ cytosolic concentrations. Nevertheless, the growth of the transgenic plants was not significantly affected by high salinity, suggesting that K⁺ nutrition was not compromised in our experiments. It should be noted that we have used a high level of K⁺ (6.5 mM) in our solutions. It would be interesting to determine the tolerance of the transgenic Brassica plants overexpressing AtNHX1 in conditions of low K⁺ availability. Transgenic plants grown in 200 mM NaCl displayed a six-fold increase in proline content compared to plants grown in low salinity. This accumulation of proline in response to high salinity is well documented. Proline contributes to osmotic adjustment, the protection of macromolecules during dehydration, and as a hydroxyl radical scavenger. (LeRudulier, et al. (1984); Yancey, et al. (1982) and Smirnoff, et al. (1989)) Evidence supporting the role of proline during salt stress was obtained on the basis of salt tolerance in transgenic tobacco plants with enhanced levels of proline biosynthesis and salt tolerance of Arabidopsis with suppressed levels of proline degradation. (Kishoor, et al. (1995) and Nanjo (1999) Moreover, a similar increase in proline content was observed in transgenic tomato plants overexpressing AtNHX1 growing at high salinity.

In all plant cells there are two major sites of lipid synthesis and desaturation of fatty acids. Glycerolipids derived from diacylglycerols synthesized in the extraplastidic compartments of the cell are synthesized by the eukaryotic pathway, whereas lipids derived from diacylglycerol synthesized in plastids are produced by a prokaryotic pathway. (Browse, et al. (1991) and Williams, et al. (2000)) Each compartment possesses different isoforms of glycerol-3-phosphate acyltransferase (GPAT) and lysophosphatidic acid acyltransferase (LPAT) that show differing specificity toward the fatty acid esterified to the two sn positions of the diacylglycerol. In addition, the desaturases of these diacylglycerol are specific to the specific compartment. Thus, through analyses of fatty acid composition it is possible to determine any specific effect of stress on lipid synthesis in the cell compartments. Our data suggest that the major structural lipids of the extraplastidic compartments (PC and PE) and of the chloroplasts (DGDG and MGDG) were unaffected by the overexpression of AtNHX1 and by the growth of the transgenic plants at high salinity. Only minor changes in the chloroplast lipids, SQDG and PG, were seen in transgenic plants grown in 200 mM NaCl. Little differences in the quantity of lipid or fatty acids were detected in the structural lipids of the cell. The 16/18 C ratio remained similar, suggesting little effect on GPAT or LPAT activities. Further, the levels of unsaturation remained constant, indicating little or no effect on the desaturase activity. Only in the minor chloroplast lipids were changes in desaturation seen, the major difference being the 16:0/trans16:1 ratio in PG (1.7 and 1.0 in transgenic plants grown in 200 mM NaCl and plants grown in low salinity, respectively). Previous work has shown that this difference reflects a change in the light-harvesting complexes of the thylakoid membranes during the acclimation of plants to stress. (Huner, et al. (1987)) Our results would suggest that the transgenic plants displayed little signs of stress or acclimation to high NaCl conditions. Analyses of the seed oil show no significant difference between seeds from wild-type and transgenic plants grown at low or high salinity.

Worldwide, more than 60 million hectares of irrigated land (representing 25% of the total irrigated acreage in the world) have been damaged by salt. (Ghassemi, et al. (1995)) Twenty years ago, Epstein argued for the development of salt tolerant crops with truly halophytic responses to salinity, i.e., accumulation of salt, in which the consumable part is botanically a fruit, such as grain or berries or pomes. (Epstein (1983)) In these plants, Na⁺ ions would accumulate mainly in their leaves, and since the water transport to the fruits and seeds is mainly symplastic (33,34,35) much of the salt from these organs would be screened. (Ehret, et al. (1986); Lee (1986) and Davies, et al. (2000)) Our results clearly support Epstein's argument. Recently, we have shown that when transgenic tomato plants, overexpressing AtNHX1, were grown at high salinity, salt accumulated in the leaves and not in the fruits. (Zhang, et al. (2001)) These results together with the data presented here clearly demonstrate the feasibility of generating salt tolerant crops for agricultural use. Much of the effort towards breeding crop cultivars with improved salt tolerance assumed that salt tolerance will be achieved only after pyramiding several characteristics in a single genotype. (Yeo, et al. and Cuartero, et al. (1999)) However, the modification of a single trait (vacuolar Na⁺ accumulation) significantly improved the salinity tolerance of Brassica plants. These results strongly suggest that with a combination of breeding and transgenic plants it could be possible to produce salt tolerant crops with far fewer introduced traits than had been anticipated.

The patent or application file contains at least one drawing executed in color. Copies of this patent or patent application publication with color drawing(s) will be provided by the Office upon request and payment of the necessary fee.

### References

All references cited herein are hereby incorporated by reference.
Maathuis, F J M. & Amtmann, A. (1999) Ann. Bot. 84,123-133.
Flowers, T J, Troke, P F & Yeo, A R (1977) Annu. Rev. Plant Physiol. 28, 89-21.
Glenn, E., Brown, J. J. & Blumwald, E. (1999) Crit. Rev. Plant Sci. 18, 227-255.
Blumwald, E., Aharon, G. S. & Apse, P. (2000) Biochim. Biophys. Acta 1465,140-151.
Shi, H., Ishitani, M., Kim, C. & Zhu, J-K. (2000). Proc. Natl. Acad. Sci. USA 97,6896-6901.
Blumwald, E.& Poole, R. J. (1985) Plant Physiol. 78, 163-167.
Apse, M. P., Aharon, G. S., Snedden, W. S. & Blumwald, E. (1999) Science 285, 1256-1258.
Ben Raïs,L., Alpha, M.-J., Bahl, J., Guillot-Salomon, T. & Dubacq, J.-P. (1993) Plant. Physiol. Biochem. 31, 547-557
Wu, J., Seliskar, D. M. & Gallagher, J. L. (1998) Physiol. Plant. 102, 307-317
Yu, B., Gong, H. & Lui, Y. (1998) J. Plant Nutrition 21, 1589-1600
Smaoui, A. & Cherif, A. (2000) Biochem. Soc. Trans. 28, 902-905
Zhang, H-X & Blumwald, E. (2001) Nature Biotechnol. 19, 765-768
Jefferson, R. A., Kavanagh, T. A. & Bevan, M. W. (1987) EMBO J. 6,3901-3907.
Moloney, M. M., Walker, J. M., & Sharma, K. K. (1989). Plant Cell Rep. 8, 238-242
Dubois, M., Gilles, R A., Hamilton, J K., Roberts, P A. & Smith, F. (1956) Anal. Chem. 28,350-356.
Bates, L. S., Waldren, R. P. & Teare, I. D. (1973) Plant & Soil 39, 205-207.
Williams, J. P. & P. A. Merrilees. (1970) Lipids 5, 367-370.
Khan, M. & J. P. Williams. (1977) J. Chromatog. 140, 79-185
Khan, M. U. & J. P. Williams (1993) Lipids 28, 953-955
Marschner,H. (1995). Mineral nutrition of higher plants. (Academic Press, New York)
Wyn Jones, R G and Pollard, A (1983). in Encyclopedia of Plant Physiology, New Series Vol 15B, eds, Lauchli, A.& Bieleski, R. L (Springer-Verlag, Berlin), pp. 528-562,
Elzam, O E. & Epstein, E. (1969). Agrochim. 13,187-195.
LeRudulier, D., Strom, A. R., Dandekar, A. M., Smith, L. T. & Valentine, R. C. (1984) Science 224,1064-1068.
Yancey, P., Clark, M., Hand, S., Bowlus, R. & Somero, G. (1982) Science 217,1214-1222.
Smirnoff, N. & Cumbes, Q. J. (1989). Phytochemistry 28,1057-1060.
Kishor, P. B. K, Hong, Z., Miao, G-H., Hu, C-A. A. & Verma, D P S. (1995). Plant Physiol. 108,1387-1394.
Nanjo, T., Kobayashi, M., Yoshiba, Y., Kakubari, Y., Yamaguchi-Shinozaki, K. & Shinozaki, K. (1999) FEBS Lett. 461,205-210.
Browse, J. A. and Somerville, C. R. (1991) Annu. Rev. Plant Physiol. Plant Mol. Biol. 42, 467-506.
Williams, J. P., V. Imperial, M. U. Khan & J. N. Hodson (2000). Biochem. J. 349, 127-133
Huner, N. P. A., M. Krol, J. P. Williams, E. Maissan, P. S. Low, D. Roberts & J. E. Thompson. (1987). Plant Physiol. 84, 12-18.
Ghassemi, F., Jakeman, A. & Nix, H. (1995) Salinization of land and water resources: Human causes, extent, management and case studies. (University of South Wales Press, Sydney).
Epstein, E. (1983) in Better Crops for Food, Ciba Foundation Symposium, eds Nugent, J. & O'Connor, M. (Pitman, London), pp. 97, 61-82.
Ehret, D. L. & Ho, L. C. (1986) J. Exp. Bot. 37,1294-1302).
Lee, D. R. A (1986) Can J. Bot. 67,1898-1902.
Davies, W. J., Bacon, M. A., Thompson, D. S., Sobeih, W. & Rodriguez, L. G. (2000) J. Exp. Bot. 51,1617-1626.
Yeo, A. R., Yeo, M. E., Flowers, S. A. & Flowers, T. J. Teor. Appl. Genet. 79, 377-384.
Cuartero, J. & Fernandez-Muñoz, R. (1999) Sci. Hort. 78, 83-125.

Zarrouk, M. (1999) Doctoral Thesis, d'Etat es-Sciences, Faculté des Sciences de Tunis.

TABLE I
Plant and seed yield of wild-type (WT) plants grown in the presence
of 10 mM NaCl and transgenic plants overexpressing AtNHX1
(X1OE₁) grown in the presence of 10 mM and 200 mM NaCl.
Each value is the Mean ± SD (n = 15).

| | WT | X1OE₁ | |
|---|---|---|---|
| | (10 mM NaCl) | (10 mM NaCl) | (200 mM NaCl) |
| Height(cm) | 210 ± 15 | 218 ± 13 | 183 ± 17 |
| Fresh Weight (g) | 1,750 ± 103 | 1,790 ± 110 | 1,630 ± 134 |
| Seeds per plant | 470 ± 39 | 481 ± 43 | 463 ± 35 |

TABLE II
Total lipid content of leaves and roots from wild-type (WT) plants
grown in the presence of 10 mM NaCl and transgenic plants over-
expressing AtNHX1 (X1OE₁) grown in the presence of 10 mM
and 200 mM NaCl. Each value is the Mean ± SD (n = 5).

| | LIPID | | | |
|---|---|---|---|---|
| | (nmole/ | WT | X1OE₁ | |
| TISSUE | gFW) | (10 mM NaCl) | (10 mM NaCl) | (200 mM NaCl) |
| LEAVES | PC | 1,120 ± 538 | 1,343 ± 375 | 1,160 ± 287 |
| | PE | 670 ± 255 | 814 ± 274 | 590 ± 214 |
| | SQDG | 403 ± 103 | 532 ± 109 | 591 ± 72 |
| | PG | 899 ± 70 | 830 ± 181 | 776 ± 158 |
| | DGDG | 1,640 ± 360 | 1,776 ± 289 | 1,817 ± 329 |
| | MGDG | 4,411 ± 532 | 4,316 ± 786 | 3,658 ± 749 |
| ROOTS | PC | 844 ± 106 | 688 ± 60 | 826 ± 88 |
| | PE | 690 ± 110 | 629 ± 60 | 660 ± 56 |
| | MQDG | 394 ± 92 | 563 ± 83 | 633 ± 50 |

TABLE III

| | | | | |
|---|---|---|---|---|
| SEQ | PROTEIN | | PROTEIN | |
| ID | NUMBER | PROTEIN | DESCRIPTION | |
| NO | (GI) | ACCESSION | (SPECIES) | SEQUENCE |

| | | | | | |
|---|---|---|---|---|---|
| 2 | NHX1 | AAD16946 | NHX1 | 1 | MLDSLVSKLP SLSTSDHASV VALNLFVALL CACIVLGHLL EENRWMNESI TALLIGLGTG |
| | 4324597 | | Na+/H+ | 61 | VTILLISKGK SSHLLVFSED LFFIYLLPPI IFNAGFQVKK KQFFRNFVTI MLFGAVGTII |
| | | | exchanger | 121 | SCTIISLGVT QFFKKLDIGT FDLGDYLAIG AIFAATDSVC TLQVLNQDET PLLYSLVFGE |
| | | | Arabidopsis | 181 | GVVTDATSVV VFNAIQSFDL THLNHEAAFH LLGNFLYLFL LSTLLGAATG LISAYVIKKL |
| | | | thaliana | 241 | YFGRHSTDRE VALMMLMAYL SYMLAELFDL SGILTVFFCG IVMSHYTWHN VTESSRITTK |
| | | | | 301 | HTFATLSFLA ETFIFLYVGM DALDIDKWRS VSDTPGTSIA VSSILMGLVM VGRAAFVFPL |
| | | | | 361 | SFLSNLAKKN QSEKINFNMQ VVIWWSGLMR GAVSMALAYN KFTRAGHTDV RGNAIMITST |
| | | | | 421 | ITVCLFSTVV FGMLTKPLIS YLLPHQNATT SMLSDDNTPK SIHIPLLDQD SFIEPSGNHN |
| | | | | 481 | VPRPDSIRGF LTRPTRTVHY YWRQFDDSFM RPVFGGRGFV PFVPGSPTER NPPDLSKA |
| 3 | 10716129 | BAB16380 | Na+/H+ | 1 | MAFGLSSLLQ NSDLFTSDHA SVVSMNLFVA LLCACIVLGH LLEENRWVNE SITALIIGLC |
| | | | exchanger | 61 | TGVVILLLSG GKSSHLLVFS EDLFFIYLLP PIIFNAGFQV KKKQFFVNFM TIMLFGAIGT |
| | | | Ipomoea | 121 | LISCSIISFG AVKIFKHLDI DFLDFGDYLA IGAIFAATDS VCTLQVLSQD ETPLLYSLVF |
| | | | nil | 181 | GEGVVNDATS VVLFNAIQSF DMTSFDPKIG LHFIGNFLYL FLSSTFLGVG IGLLCAYIIK |
| | | | | 241 | KLYFGRHSTD REVALMMLMS YLSYIMAELF YLSGILTVFF CGIVMSHYTW HNVTESSRVT |
| | | | | 301 | TRHSFATLSF VAETFIFLYV GMDALDIEKW KFVKNSQGLS VAVSSILVGL ILVGRAAFVF |
| | | | | 361 | PLSFLSNLAK KNSSDKISFR QQIIIWWAGL MRGAVSIALA YNKFTTSGHT SLHENAIMIT |
| | | | | 421 | STVTVVLFST VVFGLMTKPL INLLLPPHKQ MPSGHSSMTT SEPSSPKHFT VPLLDNQPDS |
| | | | | 481 | ESDMITGPEV ARPTALRMLL RTPTHTVHRY WRKFDDSFMR PVFGGRGFVP FVAGSPVEQS |
| | | | | 541 | PR |
| 4 | 14039961 | AAK53432 | Na+/H+ | 1 | MLSQLSSFFA SKMDMVSTSD HASVVSMNLF VALLRGCIVI GHLLEENRWM NESITALLIG |
| | | | Antiporter | 61 | LSTGIIILLI SGGKSSHLLV FSEDLFFIYL LPPIIFNAGF QVKKKQFFRN FITIILFGAV |
| | | | Suaeda | 121 | GTLVSFIIIS LGSIAIFQKM DIGSLELGDL LAIGAIFAAT DSVCTLQVLN QDETPLLYSL |
| | | | maritima | 181 | VFGEGVVNDA TSVVLFNAIQ NFDLTHIDHR IAFQFGGNFL YLFFASTLLG AVTGLLSAYV |
| | | | subsp. | 241 | IKKLYFGRHS TDREVALMML MAYLSYMLAE LFYLSGILTV FFCGIVMSHY TWHNVTESSR |
| | | | salsa | 301 | VTTKHAFATL SFVAEIFIFL YVGMDALDIE KWRFVSDSPG TSVAVSSILL GLHMVGRAAF |
| | | | | 361 | VFPFAFLMNL SKKSNSEKVT FNQQIVIWWA GLMKSAVSVA LAYNQFSRSG HTQLRGNAIM |
| | | | | 421 | ITSTITVVLF STMVFGLLTK PLILFMLPQP KHFTSASTVS DLGSPKSFSL PLLEDRQDSE |
| | | | | 481 | ADLGNDDEEA YPRGTIARPT SLRMLLNAPT HTVHHYWRRF DDYFMRPVFG GRGFVPFVPG |
| | | | | 541 | SPTEQSITNF VTENIS |
| 5 | 14211574 | BAB56105 | Na+/H+ | 1 | MAFDFGTLLG NVDRLSTSDH QSVVSIWLFV ALICACIVIG HLLEENRWMN ESITALVIGS |
| | | | Antiporter | 61 | CTGIVILLIS GGKNSHILVF SEDLFFIYLL PPIIFNAGFQ VKKKSFFRNF STIMLFGALG |
| | | | Petunia x | 121 | TLISFIIISL GAIGIFKKMN IGSLEIGDYL AIGAIFSATD SVCTLQVLNQ DETPLLYSLV |
| | | | hybrida | 181 | FGEGVVNDAT SVVLFNAIQN FDLSHIDTGK AMELVGNFLY LFASSTALGV AAGLLSAYII |
| | | | | 241 | KKLYFGRHST DREVAIMILM AYLSYMLAEL FYLSAILTVF FSGIVMSHYT WHNVTESSRV |
| | | | | 301 | TTKHTFATLS PIAEIFIFLY VGMDALDIEK WKFVSDSPGI SVQVSSILLG LVLVGRAAFV |
| | | | | 361 | FPLSFLSNLT KKTPEAKISF NQQVTIWWAG LMRGAVSMAL AYNQFTRGGH TQLRANAIMI |
| | | | | 421 | TSTITVVLFS TVVFGLMTKP LIRILLPSHK HLSRMISSEP TTPKSFIVPL LDSTQDSEAD |
| | | | | 481 | LERHVPRPHS LRMLLSTPSH TVHYYWRKFD NAFMRPVFGG RGFVPFAPGS PTDPVGGNLQ |
| 6 | 14211578 | BAB56107 | Na+/H+ | 1 | MGFESVIKLA ASETDNLWSS GHGSVVAITL FVTLLCTCIV IGHLLEENRW MNESIIALII |
| | | | Antiporter | 61 | GLATGVIILL ISGGKSSHLL VFSEDLFFIY ALPPIIFNAG FQVKKKSFFR NFATIMMFGA |
| | | | Torenia | 121 | VGTLISFIII SLGTIAFFPK MNMRLGVGDY LAIGAIFAAT DSVCTLQVLS QDETPLLYSL |
| | | | hybrida | 181 | VFGEGVVNDA TSVVLFNAVQ NFDLPHMSTA KAFELVGNFF YLFATSTVLG VLTGLLSAYI |
| | | | | 241 | IKKLYFCRHS TDREVAIMIL MAYLSYMLAE LFDLSGILTV FFCGIVMSHY TWHNVTENSR |
| | | | | 301 | VTTKHTFATL SFVAEIFIFL YVGMDALDIE KWRFVSGSMT TSAAVSATLL GLVLLSRAAP |
| | | | | 361 | VFPLSFLSNL AKKSPLEKIS LRQQIIIWWA GLMRGAVSMA LAYKQFTREG LTVERENAIF |
| | | | | 421 | ITSTITIVLF STVVFGLMTK PLINLLIPSP KLNRSVSSEP LTPNSITIPL LGESQDSVAE |
| | | | | 481 | LFSIRGQTSQ GGEPVARPSS LRMLLTKPTH TVHYYWRKFD NAFMRPVFGG RGFVPYVPGS |
| | | | | 541 | PTERSVRNWE EETKQ |
| 7 | 14488270 | BAB60901 | Na+/H+ | 1 | MAFGLSSLLQ NSELFTSDHA SVVSMNLFVA LLCACIVLGH LLEENRWVNE SITALIIGLC |
| | | | exchanger | 61 | TGVVILLLSR GKSSHLLVFS EDLFFIYLLP PIIFNAGFQV KKKQFFVNFM TIMLFGAIGT |
| | | | Ipomoea | 121 | LISCSIISFG AVKIFKHLDI DFLDFGDYLA IGAIFAATDS VCTLQVLSQD ETPLLYSLVF |
| | | | tricolor | 181 | GEGVVNDATS VVLFNAIQSF DMTSFDPKIG LHFIGNFLYL FLSSTFLGVG IGLLCAYIIK |
| | | | | 241 | KLYFGRHSTD REVALMMLMS YLSYIMAELF YLSGILTVFF CGIVKSHYTW HNVTESSRVT |
| | | | | 301 | TRHSFATLSF VARTFIFLYV GMDALDIEKW KFVKNSQGLS VAVSSILVGL ILVGRAAFVF |
| | | | | 361 | PLSFLSNLAK KNSSDKISFR QQIIIWWAGL MRGAVSIALA YNKFTTSGHT SLHENAIMIT |
| | | | | 421 | STVTVVLFST VVFGLMTKPL INLLLPPHKQ IASGHSSMTT SEPSSPKHFA VPLLDNQHDS |
| | | | | 481 | ESDMITGPEV ARPTALRMLL RTPTHTVHRY WRKFDDSFMR PVFGGRGFVP FVAGSPAEQS |
| | | | | 541 | PR |
| 8 | 4585981 | AAD25617 | similar to | 1 | MISPVEHDPQ GQVKQQQAAG VGILLQIMML VLSFVLGHVL RRHRFHYLPE ASGSLLIGLI |
| | | | Na+/H+− | 61 | VGILANISDT ETSIRFCPPP SIPEFSLLSF PRSLVCSFYS VSGRGLISTK SSSSCFCCLP |
| | | | exchanging | 121 | SYYILCFNIC ISSFKFAAAM LCINDVIFLD IIHLFEPSQV SVFNLNHSFL TLEPLLPLLS |
| | | | proteins | 181 | SELLSLQLLL VVCYLGGSMY LMYKLPFVEC LMFGALISAT DPVTVLSIFQ VLLLFLLLSV |
| | | | Arabidopsis | 241 | STGYKYSHDV GTDVNLYALV FGESVLNDAV SFYYLLRYWA LPFKTMSLVN RQSSSGEHFF |
| | | | thaliana | 301 | MVVIRFFETF AGSMSAGLAI SFLNSFYTVV FTLLILSEHI VNVMSLFSLF STSIHACRRC |
| | | | | 361 | WSLRHCFYTL HRNCNRRVMK RYTFSNLSEA SQSFVSSFFH LISSLAETFT FIYMGFDIAM |
| | | | | 421 | EQHSWSHVGA VNVFGCAYLV NLFRQENQKI PMKHQKALWY SGLRGAMAFA LALQSLHDLP |
| | | | | 481 | EGHGQIIFTA TTTIVVVTVT FVLLIGGSTG KMLEALEVVG DDLDDSMSEV NSRRSTLISL |
| | | | | 541 | NIGASSDEDT SSSGSRFKMK LKEFHKTGDG DGDGE |
| 9 | 8515714 | AAF76139 | putative | 1 | MTTVIDATMA YRFLEEATDS SSSSSSSKLE SSPVDAVLFV GMSLVLGIAS RHLLRGTRVP |
| | | | Na+/H+ | 61 | YTVALLVIGI ALGSLEYGAK HNLGKIGHGI RIWNEIDPEL LLAVFLPALL FESSFSMEVH |
| | | | antiporter | 121 | QIKRCLGQMV LLAVPGVLIS TACLGSLVKV TFPYEWDWKT SLLLGGLLSA TDPVAVVALL |
| | | | SOS1 | 181 | KELGASKKLS TIIEGESLMN DGTAIVVFQL FLKMAMGQNS DWSSIIKFLL KVALGAVGIG |
| | | | Arabidopsis | 241 | LAFGIASVIW LKFIFNDTVI EITLTIAVSY FAYYTAQEWA GASGVLTVMT LGMFYAAFAR |
| | | | thaliana | 301 | TAFKGDSQKS LHHFWEMVAY IANTLIFILS GVVIAEGILD SDKIAYQGNS WRFLFLLYVY |
| | | | | 361 | IQLSRVVVVG VLYPLLCRFG YGLDWKESII LVWSGLRGAV ALALSLSVKQ SSGNSHISKE |
| | | | | 421 | TGTLFLFFTG GIVFLTLIVN GSTTQFVLRL LRMDILPAPK KRILEYTKYE MLNKALRAFQ |
| | | | | 481 | DLGDDEELGP ADWPTVESYI SSLKGSEGEL VHHPHNGSKI GSLDPKSLKD IRMRFLNGVQ |
| | | | | 541 | ATYWEMLDEG RISEVTANIL MQSVDEALDQ VSTTLCDWRG LKPHVNFPNY YNFLHSKVVP |
| | | | | 601 | RKLVTYFAVE RLESACYISA AFLRAHTIAR QQLYDFLGES NIGSIVINES EKEGEEAKKF |
| | | | | 661 | LEKVRSSFPQ VLRVVKTKQV TYSVLNHLLG YIENLEKVGL LEEKEIAHLH DAVQTGLKKL |
| | | | | 721 | LRNPPIVKLP KLSDMITSHP LSVALPPAFC EPLKHSKKEP MKLRGVTLYK EGSKPTGVWL |
| | | | | 781 | IFDGIVKWKS KILSNNHSLH PTFSHGSTLG LYEVLTGKPY LCDLITDSMV LCFFIDSEKI |
| | | | | 841 | LSLQSDSTID DFLWQESALV LLKLLRPQIF ESVAMQELRA LVSTESSKLT TYVTGESIEI |
| | | | | 901 | DCNSIGLLLE GFVKPVGIKE ELISSPAALS PSNGNQSFHN SSEASGIMRV SFSQQATQYI |
| | | | | 961 | VETRARAIIF NIGAFGADRT LHRRPSSLTP PRSSSSDQLQ RSFRKEHRGL MSWPENIYAK |
| | | | | 1021 | QQQEINKTTL SLSERAMQLS IFGSMVNVYR RSVSFGGIYN NKLQDNLLYK KLPLNPAQGL |
| | | | | 1081 | VSAKSESSIV TKKQLETRKH ACQLPLKGES STRQNTMVES SDEEDEDEGI VVRIDSPSKI |
| | | | | 1141 | VFRNDL |
| 10 | 9857314 | BAB11940 | Na/H | 1 | MWSQLSSLLS GKMDALTTSD HASVVSMNLF VALLCGCIVI GHLLEENRWM NESITALLIG |
| | | | antiporter | 61 | LATGVVILLI SGGKSSHLLV FSEDLFFIYL LPPIIFNAGF QVKKKQFFRN FITIVLFGAV |
| | | | Nhx1 | 121 | GTLVSFTIIS LGALSIFKKL DIGTLELADY LAIGAIFAAT DSVCTLQVLN QDETPLLYSL |
| | | | Atriplex | 181 | VFGEGVVNDA TSVVLFNAIQ SFDLTRIDHR IALQFMGNFL YLFIASTILG AFTGLLSAYI |
| | | | gmelini | 241 | IKKLYFGRHS TDREVALMML MAYLSYMLAE LFYLSGILTV FFCGIVMSHY TWHNVTESSR |
| | | | | 301 | VTTKHAFATL SFVAEVFLFL YVGMDALDIE KWRFVSDSPG ISVAVSSILL GLVMVGRAAF |
| | | | | 361 | VFPLSWLMNF AKKSQSEKVT FNQQIVIWWA GLMRGAVSMA LAYNQFTRSG HTQLRGNAIM |
| | | | | 421 | ITSTISVVLF STMVFGLLTK PLIMFLLPQP KHFTSCSTVS DVGSPKSYSL PLLEGNQDYE |
| | | | | 481 | VDVCNGNHED TTEPRTIVRP SSLRMLLNAP THTVHHYWRK FDDSFMRPVF GGRGFVPFVP |
| | | | | 541 | GSPTEQSTNN LVDRT |
| 11 | NHA1 | NP_013239 | Putative | 1 | MAIWEQLEVS KAHVAYACVG VFSSIFSLVS LYVKEKLYIG ESTVAGIFGL IVGPVCLNWF |
| | 6323167 | | Na+/H+ | 61 | NPLKWGNSDS ITLEITRIVL CLQIFAVAVE LPRKYMLKHW VSVTMLLLPV MTAGWLIIGL |
| | | | antiporter, | 121 | FVWILIPGLN FSASLLISAC ITATDPILAQ SVVSGKFAQR VPGHLRNLLS AESGCNDGMA |
| | | | Nhalp | 181 | FPFLFLSMNL ILHPGNGREI VKDWICVTIL YECLFGCLLG CFIGYVGRIT IRFAEKKNII |
| | | | Saccharo- | 241 | DRESFLAFYV VLAFMCAGFG SILGVDDLLV SFAAGATFAW DGWFSQKTQE SNVSTVIDLL |
| | | | myces | 301 | LNYAYFIYFG AIIPWSQFNN GEIGTNVWRL IILSIVVIFL RRIPAVNILR PLIPDIKSWR |
| | | | cerevisiae | 361 | EALFVGHFGP IGVGAIFAAI LARGELESTF SDEPTPLNVV PSKEESKHWQ LIACIWPITC |
| | | | | 421 | FFIVTSIIVH GSSVAIITLG RHLNTITLTK TFTTHTTNGD NGKSSWMQRL PSLDKAGRSF |
| | | | | 481 | SLHRMDTQMT LSGDEGEAEE GGGRKGLAGG EDEEGLNNDQ IGSVATSGIP ARPAGGMPRR |
| | | | | 541 | RKLSRKEKRL NRRQKLRNKG REIFSSRSKN EMYDDDELND LGRERLQKEK EARAATFALS |
| | | | | 601 | TAVNTQRNEE IGMGGDEEED EYTPEKEYSD NYNNTPSFES SERSSSLRGR TYVPRNRYDG |
| | | | | 661 | EETESEIESE DEMENESERS MASSEERRIR KMKEEEMKPG TAYLDGNRNI IENKQGEILN |
| | | | | 721 | QVDIEDRNEA RDDEVSVDST AHSSLTTTMT NLSSSSGGRL KRILTPTSLG KIHSLVDKGK |
| | | | | 781 | DKNKNSKYHA FKIDNLLIIE NEDGDVIKRY KINPHKSDDD KSKNRPRNDS VVSRALTAVG |
| | | | | 841 | LKSKANSGVP PPVDEEKAIE GPSRKGPGML KKRTLTPAPP RGVQDSLDLE DEPSSEEDLG |
| | | | | 901 | DSYNMDDSED YDDNAYESET EFERQRRLNA LGEMTAPADQ DDEELPPLPV EAQTGNDGPG |
| | | | | 961 | TAEGKKKQKS AAVKSALSKT LGLNK |
| 12 | NHX1 | NP_010744 | Required | 1 | MLSKVLLNIA FKVLLTTAKR AVDPDDDDEL LPSPDLPGSD DPIAGDPDVD LNPVTEEMFS |
| | 6320663 | | for intra- | 61 | SWALFIMLLL LISALWSSYY LTQKRIRAVH ETVLSIFYGM VIGLIIRMSP GHYIQDTVTF |
| | | | cellular | 121 | NSSYFFNVLL PPIILNSGYE LNQVNFFNNM LSILIFAIPG TFISAVVICI ILYIWTFLGL |
| | | | sequestra- | 181 | ESIDISFADA MSVGATLSAT DPVTILSIFN AYKVDPKLYT IIFGESLLND AISIVMFETC |
| | | | tion of | 241 | QKFHGQPATF SSVFEGAGLF LMTFSVSLLI GVLIGILVAL LLKHTHIRRY PQIESCLILL |
| | | | Na+; Nhx1p | 301 | IAYESYFFSN GCHMSGIVSL LFCGITLKHY AYYNMSRRSQ ITIKYIFQLL ARLSENFIFI |
| | | | Saccharo- | 361 | YLGLELFTEV ELVYKPLLII VAAISICVAR WCAVFPLSQF VNWIYRVKTI RSMSGITGEN |
| | | | myces | 421 | ISVPDEIPYN YQMMTFWAGL RGAVGVALAL GIQGEYKFTL LATVLVVVVL TVIIFGGTTA |
| | | | cerevisiae | 481 | GMLEVLNIKT GCISEEDTSD DEFDIEAPRA INLLNGSSIQ TDLGPYSDNN SPDISIDQFA |
| | | | | 541 | VSSNKNLPNN ISTTGGNTFG GLNETENTSP NPARSSMDKR NLRDKLGTIF NSDSQWFQNF |
| | | | | 601 | DEQVLKPVFL DNVSPSLQDS ATQSPADFSS QNH |
| 13 | NHX2 | NP_187154 | NHX2 | 1 | MTMFASLTSK MLSVSTSDHA SVVSLNLFVA LLCACIVIGH LLEENRWMNE SITALLIGLG |
| | 15229877 | | Na+/H+ | 61 | TGVVILLISR GKNSHLLVFS EDLFFIYLLP PIIFNAGPQV KKKQFFRNFV TIMAFGAIGT |
| | | | exchanger | 121 | VVSCTIISLG AIQFFKKLDI GTFDLGDFLA IGAIFAATDS VCTLQVLNQD ETPLLYSLVF |
| | | | Arabidopsis | 181 | GEGVVNDATS VVLFNAIQSF DLTHLNHEAA FQFLGNFFYL FLLSTGLGVA TGLISAYVIK |
| | | | thaliana | 241 | KLYFGRHSTD REVALMMLMA YLSYMLAELF ALSGILTVFF CGIVMSHYTW HNVTESSRIT |
| | | | | 301 | TKHAFATLSF LAETFIFLYV GMDALDIEKW RFVSDSPGTS VAVSSILMGL VMLGRAAPVF |
| | | | | 361 | PLSFLSNLAK KHQSEKISIK QQVVIWWAGL MRGAVSMALA YNKFTRSGHT ELRGNAIMIT |
| | | | | 421 | STITVCLFST MVFGMLTKPL IRYLMPHQKA TTSTTSMLSD DSTPKSIHIP LLDGEQLDSF |
| | | | | 481 | ELPGSHQDVP RPNSLRGFLM RPTRTVHYYW RQFDDAFMRP VFGGRGFVPF VPGSPTERSS |
| | | | | 541 | HDLSKP |
| 14 | NHX3 | NP_200358 | NHX3 | 1 | MSIGLTEFVT NKLAAEHPQV IPISVFIAIL CLCLVIGHLL EENRWVNESI TAILVGAASG |
| | 15240159 | | Na+/H+ | 61 | TVILLISKGK SSHILVFDEE LFFIYLLPPI IFNAGFQVKK KKFFHNFLTI MSFGVIGVFI |
| | | | exchanger | 121 | STVIISFGTW WLFPKLGFKG LSARDYLAIG TIFSSTDTVC TLQILHQDET PLLYSLVFGE |
| | | | Arabidopsis | 181 | GVVNDATSVV LFNAVQKIQF ESLTGWTALQ VFGNFLYLFS TSTLLGIGVG LITSFVLKTL |
| | | | thaliana | 241 | YFGRHSTTRE LAIMVLMAYL SYMLAELFSL SGILTVFFCG VLMSHYASYN VTESSRITSR |
| | | | | 301 | HVFAMLSFIA ETFIFLYVGT DALDFTKWKT SSLSFGGTLG VSGVITALVL LGRAAFVFPL |
| | | | | 361 | SVLTNFMNRH TERNESITFK HQVIIWWAGL MRGAVSIALA FKQFTYSGVT LDPVNAAMVT |
| | | | | 421 | NTTIVVLFTT LVFGFLTKPL VNYLLPQDAS HNTGNRGKRT EPGSPKEDAT LPLLSFDESA |
| | | | | 481 | STNFNRAKDS ISLLMEQPVY TTHRYWRKFD DTYMRPIFGG PRRENQPEC |
| 15 | NHX4 | NP_187288 | NHX4 | 1 | MVIGLSTMLE KTEALFASDH ASVVSMNLFV ALLCACIVLG HLLEETRWMN ESITALIIGS |
| | 15230706 | | Na+/H+ | 61 | CTGIVILLIS GGKSSRILVF SEDLFFIYLL PPIIFNAGFQ VKKKQFFRNF MTIMLFGAIG |
| | | | exchanger | 121 | TLISFVIISF GAKHLFEKMN IGDLTIADYL AIGAIFSATD SVCTLQVLNQ DETPLLYSLV |
| | | | Arabidopsis | 181 | FGEGVVNDAT SVVLFNAIQR FDLTNINSAI ALEFAGNFFY LFILSTALGV AAGLLSAFVI |
| | | | thaliana | 241 | KKLYIGRHST DREVALMMLL AYLSYMLAEL FHLSSILTVF FCGIVMSHYT WHNVTDKSKV |
| | | | | 301 | TTKHTFAAMS FLAEIFIFLY VGMDALDIEK WDVVRNSPGQ SIGVSSILLG LILLGRAAFV |
| | | | | 361 | FPLSFLSNLT KSSPDEKIDL KKQVTIWWAG LMRGAVSMAL AYNQFTTSCH TKVLGNAIMI |
| | | | | 421 | TSTITVVLFS TVVFGLLTKP LVKHLQPSSK QSSTTALQIT LRSSFHDPIL HEPLLSTQGQ |
| | | | | 481 | SEYDPEQHVS FRMFWKSPSR AIHHYWRKFD NAVMRRIFGG RGVSPVVPGS PIENSVPQWS |
| | | | | 541 | EEVENKEQNG EP |
| 16 | NHX5 | NP_175839 | NHX5 | 1 | MEEVMISPVE HDPQGQVKQQ QAAGVGILLQ INMLVLSFVL GHVLRRHRFH YLPEASGLIV |
| | 30695721 | | Na+/H+ | 61 | GILANISDTE TSIRFCPPPS IPEFSLLSFP RSLKPFFSNF GAIVTFAIIG TFVASVVTGG |
| | | | exchanger | 121 | LVYLGGSMYL MYKLPFVECL MFGALISATD PVTVLSIFQD VGTDVNLYAL VFGESVLNDA |
| | | | Arabidopsis | 181 | VSFYYLLRYW ALPFKFFETF AGSMSAEHLF KYAGLDTENL QNLECCLFVL FPYFSYMLAE |
| | | | thaliana | 241 | GVGLSGIVSI LFTGIVMKRY TFSNLSEASQ SFVSSFFHLI SSLAETFTFI YMGFDIAMEQ |
| | | | | 301 | HSWSHVGFIL FSIVSSFTDR QAVNVFGCAY LVNLFRQENQ KIPMKHQKAL WYSGLRGAMA |
| | | | | 361 | FALALQSLHD LPEGHGQIIF TATTTIVVVT VLLIGGSTGK MLEALEVVGD DLDDSMSEGF |
| | | | | 421 | EESDHQYVPP PFSIGASSDE DTSSSGSRFK MKLKEFHKTT TSFTALDKNF LTPFFTTNSG |
| | | | | 481 | DGDGDGE |
| 17 | NHX6 | NP_178079 | NHX6 | 1 | MSSELQISPA IHDPQGQEKQ QQAAGVGILL QIMMLVLSFV LGHVLRRHKF YYLPEASASL |
| | 22330742 | | Na+/H+ | 61 | LIGLIVGGLA NISNTETSIR FVELFLISFF RHGSISTMSS SFCFCCLPSY YILKIEYLGG |
| | | | exchanger | 121 | VMFLMYRLPF VECLMFGSLI SATDPVTVLS IFQELGSDVN LYALVFGESV LNDADEIVTL |
| | | | Arabidopsis | 181 | LIRSFSFLCC FWQMAISLYR TMSLVRSHSS GQNFFMVIVR FLETFVGSMS AAMKYFILMY |
| | | | thaliana | 241 | SLLLSVYRTW SAVSSYFFHI SRNKTLLFYT SYVSIYFTLI EIVQFVMKHY TYSNLSANSQ |
| | | | | 301 | RFVSAFFHLI SSLARTFVFI YMGFDIAMEK HSWAANVFGC GYLVNLARPA HRKIPMTHQK |
| | | | | 361 | ALWYSGKILL CVPLSSYCFY SSVINTKICG FCIGLRGAMA FALALQSVHD LPEGHGQTIF |
| | | | | 421 | TATTAIVVLT VLLIGGSTGT MLEALEVVGD SHDTSLGDGF EVVNSRYMTS YDDEDTPPGS |
| | | | | 481 | GFRTKLREFH KSAASFTELD RNYLTPFFTS NNGDYDDEGN MEQHHGNNII L |
| 18 | NHX7 | NP_178307 | NHX7 | 1 | MTSIIGAALP YKSPEKAIAS SSYSAENDSS PVDAVIFAGT SLVLGTACRY LFNGTRVPYT |
| | 22325422 | | Na+/H+ | 61 | VVLLVIGIFL GSLEYGTKHN LGKLGHGIRI WNGINPDLLL AVFLPVLLFE SSFSMDVHQI |
| | | | exchanger | 121 | KRCMGQMVLL AGPGVLISTF CLGALIKLTF PYNWDWKTSL LLGGLLGATD PVAVVALLKE |
| | | | Arabidopsis | 181 | LGASKKMTTL IDGESLMNDG VSVVVFQLFF KMVMGHNSDW GSIIKFLVQN SFGAVGIGLA |
| | | | thaliana | 241 | FGIASVFWLK FIFNDTVAQI TVTLSASYFA YYTAQEWAGV SGILTVMILG MFFAAFARTA |
| | | | | 301 | FKGDSHQSLH HFWYFTTQEM AAYIANTLVF MLSGVIIAES VLSGQTISYK AIKWKFISQF |
| | | | | 361 | RYGNKAVLQF LFLTGGIVFL TLVVNGSTTQ LLLHLLRMDT LTATKKRILE YTKFEMMKTA |
| | | | | 421 | LKAFENLGDD EELGSADWPT VIRHISSLKD LEGRQVNPHD GYEAGSLDPT NIMDIRVQAA |
| | | | | 481 | YWEMLDDGRI TQCTANVLMQ SVDEALDLVS TSSLSDWRGL EPRVHFPNYY KFLQSKIIPH |
| | | | | 541 | KLVTHLIVER LESACYISSA FLRAHRIARQ QLHIFLGNSN IASTVINESE VEGEEAKQFL |
| | | | | 601 | EDVRDSFPQV LSVLKTRQVT HYVLNHLNCY IKNLEKVGLL EGKEVSHLHD VVQSDLKKLL |
| | | | | 661 | RHPPSLKLPN VDDLITSNPL LKDRSSFRSL AIGETDA |
| 19 | NHX8 | NP_172918 | NHX8 | 1 | MTSIIGAALP YKSPEKAIAS SSYSAENDSS PVDAVIFAGT SLVLGTACRY LFNGTRVPYT |
| | 15223849 | | Na+/H+ | 61 | VVLLVIGIFL GSLEYGTKHN LGKLGHGIRI WNGINPDLLL AVFLPVLLFE SSFSMDVHQI |
| | | | exchanger | 121 | KRCMGQMVLL AGPGVLISTF CLGALIKLTF PYNWDWKTSL LLGGLLGATD PVAVVALLKE |
| | | | Arabidopsis | 181 | LGASKKMTTL IDGESLMMDG VSVVVFQLFF KMVMGHNSDW GSIIKFLVQN SFGAVGIGLA |
| | | | thaliana | 241 | FGIASVFWLK FIFNDTVAQI TVTLSASYFA YYTAQEWAGV SGILTVMILG MFFAAFARTA |
| | | | | 301 | FKGDSHQSLH HFWYFTTQEM AAYIANTLVF MLSGVIIAES VLSGQTISYK AIKWKFISQF |
| | | | | 361 | RYGNKAVLQF LFLTGGIVFL TLVVNGSTTQ LLLHLLRMDT LTATKKRILE YTKFEMMKTA |
| | | | | 421 | LKAFENLGDD EELGSADWPT VIRHISSLKD LEGRQVNPHD GYEAGSLDPT NIMDIRVQAA |
| | | | | 481 | YWEMLDDGRI TQCTANVLMQ SVDEALDLVS TSSLSDWRGL EPRVHFPNYY KFLQSKIIPH |
| | | | | 541 | KLVTHLIVER LESACYISSA FLRAHRIARQ QLHIFLGNSN IASTVINESE VEGEEAKQPL |
| | | | | 601 | EDVRDSFPQV LSVLKTRQVT HYVLNHLNGY IKNLEKVGLL EGKEVSHLHD VVQSDLKKLL |
| | | | | 661 | RHPPSLKLPN VDDLITSNPL LKDRSSFRSL AIGETDA |
| 20 | 15982204 | CAC84522 | Na+/H+ | 1 | MGLDAVARLG VSILSDGDQV SVDSITLFVA LLCGCIVIGH LLEESRWIND SITTLVIGLS |
| | | | antiporter, | 61 | TGGIILLTTK GKSSHLLEFD EQLFFIYVLP PIIFNAGFQV KKKQFFRNFV TIMLFGAVGT |
| | | | isoform 1 | 121 | LISFSIISFG AKELLGKLDI GFLELRDYLA IGAIFSATDS VCTLQALNQD ETPRLYSLVF |
| | | | Lyco- | 181 | GEGVVNDATS VVLFNAIQKL DLSHINSRAA LVFTGNFLYL FLASTFLGVL ICLLSAYLIK |
| | | | persicon | 241 | KIYLGRHSTD REVALMILMA YLSYVMAELF DLSGILTVFI CGIVMSHYTW HNVTFNSKVT |
| | | | esculentum | 301 | TRHAFATLSF IAEIFIFLYV GMDALDIEKW RFVKDSPGKS VGVSAALLGL VLVGRACFVF |
| | | | | 361 | PLSLFSNCLK RSEHDKFGLK LQVTIWWAGL MRGSVSMALA YNQFTRFGHT QQPGNAVMIT |
| | | | | 421 | STITIVLFST VVFGLITKPL VRFLLPSSQG FNNLISSEQS FARPLLTNEQ ELELEMGNVD |
| | | | | 481 | PVRPSGLSIL LKEPSYTIHN HWRRFDDAFM RPLFGGRGFV PDAPELSKGG CDQY |
| 21 | 15982206 | CAC83608 | Na+/H+ | 1 | MEDHLQISPA GAKAIPGKEQ QAAGYGILLQ IMMLVLSFVI GHVLRRRHFY YIPEASASLL |
| | | | antiporter, | 61 | IGLIVGGLAN VSDTETSIRA WFNFHEEFFF LFLLPPTIFQ SGFSLSPKPF FSNFGAIITF |
| | | | isoform 2 | 121 | AILGTFIASF VTGILVYLGG VTYLMYRLPF VECLMFGALI SATDPVTVLS IFQELGTDVN |
| | | | Lyco- | 181 | LYALVFGESV LNDAMAISLY RTMSLVRSHM STDQNYFMIT IRFVETFMGS LSAGVGVGFV |
| | | | persicon | 241 | SALLFKYAGL DIDNLQNLES CLFVLFPYFS YMLAEGLGLS GIVSILFTGV VMKRYTYPNL |
| | | | esculentum | 301 | SESSQRFVSA FFHLISSLAE TFVFIYMGFD IAMEKHSWSH VGFIFFSILF IVIARAANVF |
| | | | | 361 | GCAYLVNLVR PPHQKIPAKH QKALWYSGLR GAMAFALALQ PVHDLPEGHG QAIFTATTAI |
| | | | | 421 | VVLTVLIIGG SAGTMLEALE VVGDGQSGSM DETFEGNNGY IAPSYRDESY DGEPSSGNRF |
| | | | | 481 | RMKLKEFHKS TTSFSALDKN YLTPFFTTQG GDEDEDEPIM HSSRRAGYDG H |
| 22 | 5731737 | BAA83337 | OsNHX1 | 1 | MGMEVAAARL GALYTTSDYA SVVSINLFVA LLCACIVLGH LLEENRWVNE SITALIIGLC |
| | | | Oryza | 61 | TGVVILLMTK GKSSHLFVFS EDLFFIYLLP PIIFNAGFQV KKKQFFRNFM TITLFGAVGT |
| | | | sativa | 121 | MISFFTISIA AIAIFSRMNI GTLDVGDFLA IGAIFSATDS VCTLQVLNQD ETPFLYSLVF |
| | | | (japonica | 181 | GEGVVNDATS IVLFNALQNF DLVHIDAAVV LKFLGNFFYL FLSSTFLGVF AGLLSAYIIK |
| | | | cultivar- | 241 | KLYIGRHSTD REVALMMLMA YLSYMLAELL DLSGILTVFF CGIVMSHYTW HNVTESSRVT |
| | | | group) | 301 | TKHAFATLSF IAETFLFLYV GMDALDIEKW EFASDRPGKS IGISSILLGL VLIGRAAFVF |
| | | | | 361 | PLSFLSNLTK KAPNEKITWR QQVVIWWAGL MRGAVSIALA YNKFTRSGHT QLHGNAIMIT |
| | | | | 421 | STTTVVLFST MVFGMMTKPL IRLLLPASGH PVTSEPSSPK SLHSPLLTSM QGSDLESTTN |
| | | | | 481 | IVRPSSLRML LTKPTHTVHY YWRKFDDALM RPMFGGRGFV PFSPGSPTEQ SHGGR |
| 23 | 14211576 | BAB56106 | Na+/H+ | 1 | MAFDFGTLLG KMNNLTTSDH QSVVSVNLFV ALICACIVIG HLLEENRWNN ESITALVIGS |
| | | | antiporter, | 61 | CTGVIILLIS GGKNSHILVF SEDLFFTYLL PPTIFNAGFQ VKKKSFFRNF STIMLFGAVG |
| | | | Nierem- | 121 | TLISFIIISA GAIGIFKKMD IGHLEIGDYL AIGAIFAATD SVCTLQVLNQ EETPLLYSLV |
| | | | bergia | 181 | FGEGVVNDAT SVVLFNAVQN FDLSHISTGK ALQLIGNFLY LFASSTFLGV AVGLLSAFII |
| | | | caerulea | 241 | KKLYFGRHST DREVAIMILM AYLSYMLAEL FYLSGILTVF FCGIVMSHYT WNNVTESSRV |
| | | | | 301 | TTKHTFATLS FIAEIFIFLY VGMDALDIEK WKFVSDSPGT SIKVSSILLG LVLVGRGAFV |
| | | | | 361 | FPLSFLSNLT KKNPEDKISF NQQVTIWWAG LMRGAVSMAL AYNQFTRGGH TQLRANAIMI |
| | | | | 421 | TSTITVVLFS TVVFGLMTKP LILLLLPSQK HLIRMISSEP MTPKSFIVPL LDSTQDSEAD |
| | | | | 481 | LGRHVPRPHS LRMLLSTPSH TVHYYWRKFD NAFMRPVFGG RGFVPFVPGS PTEPVEPTEP |
| | | | | 541 | RPAESRPTEP TDE |
| 24 | 15812035 | AAK27314 | Na+/H+ | 1 | MDQAISSVVR KLQMVNTSDH NSVVSINIFV ALPCASIVIG HLLEESRWNN ESITALLIGV |
| | | | exchanger | 61 | CAGVIILLTT GGKSSHLFVF SEDLFFIYVL PPIIFNAGFQ VKKKQFFRNF ITIMLFGAIG |
| | | | Citrus x | 121 | TLVSCTIISL GVIQFFKKLD IGTLDIGDYL AIGAIFAATD SVCTLQVLNQ DDTPLLYSLV |
| | | | paradisi | 181 | FGEGVVNDAT SVVLFNAIQS FDLTHINTRS AFQFIGNFLY LFFTSTLLGV IGGLLSAYVI |
| | | | | 241 | KKLYFGRHST DREVAIMVLM AYLSYMLAEL FYLSCILTVF FCGIVMSHYT WHNVTESSRV |
| | | | | 301 | TTKHTFATLS FVAEIFTFLY VGMDALDIEK WRFVKGSPGT SVAASAMLMG LIMAGRAAFV |
| | | | | 361 | FPLSFLTNLA KKSPTEKISI KQQVIIWWAG LMRGAVSMAL AYNQFTRSGH TQLRENAIMI |
| | | | | 421 | TSTITVVLFS TVVFGLMTEP LIRLLLPHPK HTTNHILSDP STPKSLSQPL LEEGQQDSYA |
| | | | | 481 | DLVGPTVPRP GSLRALLTTP THTVHYYWRK FDDAFMRPVF GGRGFAPFVP GSPTERSVRG |
| | | | | 541 | GQ |
| 25 | 15027833 | AAK76737 | Na+/H+ | 1 | MGLDLGALAL KYTGLAVSDH DSIVAINIFI ALLCGCIVFG HLLEGNRWVN ESTTALVLGL |
| | | | antiporter | 61 | ITGGVILICT KGVNSRILIF SEDIFFIYLL PPIIFNAGFQ VKKKQFFRNF ATIILFGAAG |
| | | | Triticum | 121 | TLISFVIITF GAMGLFSKLD VGPLELGDYL AIGAIFSATD SVCTLQVLNQ DEAPLLYSLV |
| | | | aestivum | 181 | FGEGVVNDAT SVVLFNAIQN IDINHFDVFV LLQFIGKFLY LFFTSTVLGV AAGLLSAYII |
| | | | | 241 | KKLCFARHST DREVAIMILM AYLSYMLSML LDLSGILTVF FCGIVMSHYT WHNVTESSRV |
| | | | | 301 | TTKHTFATLS FIAEIFLFLY VGMDALDIDK WKLASSSPKK PIALSAVILG LVMVGRAAFV |
| | | | | 361 | FPLSFLSNLS KKESHPKISF NQQVIIWWAG LMRGAVSIAL AYNKFTTSGH TAVRVNAVMI |
| | | | | 421 | TSTIIVVLFS TMVFGLLTKP LINLLIPPRP GTAADISSQS FLDPLTASLL GSDFDVGQLT |
| | | | | 481 | PQTNLQYLLT MPTRSVHRVW RKFDDKFMRP MFGGRGFVPF VPGSPIERSV HGPGLLGTVT |
| | | | | 541 | EAEDRS |
| 26 | 28575021 | AAK76738 | Na+/H+ | 1 | MGYQVVAAQL ARLSGALGTS DHASVVSITL FVALLCACIV LGHLLEENRW LNESITALII |
| | | | antiporter | 61 | GLCTGVVILM TTKGKSSHVL VFSEDLFFIY LLPPIIFNAG FQVKKKQFFR NFMAITLFGA |
| | | | Triticum | 121 | VGTMMSFFTI SLAAIAIFSR MNIGTLDVSD FLAIGAIFSA TDSVCTLQVL NQDETPFLYS |
| | | | aestivum | 181 | LVFGEGVVND ATSVVLFNAL QNFDPNQIDA IVILKFLGNF CYLFVSSTFL GVFTGLLSAY |
| | | | | 241 | VIKKLYIGRH STDREVALVM LMAYLSYMLA ELLDLSGILT VFFCGIVMSH YTWHNVTESS |
| | | | | 301 | RVTTKHAFAT LSFIAETFLF LYVGMDALDI EKWKFASDSP GKSIGISSIL LGLVLVGPAA |
| | | | | 361 | FVFPLSFLSN LTKKTELEKI SWRQQIVIWW AGLMRGAVSI ALAYNKFTRS GHTQLHGNAI |
| | | | | 421 | MITSTITVVL FSTMLFGILT KPLIRFLLPA SSNGAASDPA SPKSLHSPLL TSQLGSDLEA |
| | | | | 481 | PLPIVRPSSL RMLITKPTHT IHYYWRKFDD ALMRPMFGGR GFVPYSPGSP TDPNVLVE |
| 27 | 31580736 | AAP55209 | Na+/H+ | 1 | MGLDLGALAL KYTGLAVSDH DSIVAINTFI ALLCGCIVFG HLLGGNRWVN ESTAALVLGL |
| | | | antiporter | 61 | ITGGVILICT KGVNSRILIF SEDIFFIYLL PPIIFNAGFQ VKKKQFFRNF ATIILFGAAG |
| | | | Triticum | 121 | TLISFVIITF GAMGLFSKLD VGPLELGDYL AIGAIFSATD SVCTLQVLNQ DEAPLLYSLV |
| | | | aestivum | 181 | FGEGVVNDAT SVVLFNAIQN IDINHFDVFG LLQFIGKFLY LFFTSTVLGV AAGLLSAYII |
| | | | | 241 | KKLCFARHST DREVAIMILM AYLSCMLSML LDLSGILTVF FCGIVMSHYT WHNVTESSRV |
| | | | | 301 | TTKHTFATLS FIAEIFLFLY VGMDALDIDK WKLASSSPKK PIALSAVILG LVMVGRAAFV |
| | | | | 361 | FPLSFLSNLS KKESHPKISF NQQVIIWWAG LMRGAVSIAL AYNKFTTSGH TAVRVNAVMI |
| | | | | 421 | TSTIIVVLFS TMVFGLLTKP LINLLIPPRP GTAADISSQS FLDPLTASLL GSDFDVGQLT |
| | | | | 481 | PQTNLQYLLT MPTRSAHRVW RKFDDKFMRP MFGGRGFVPF VPGSPIERSV HGPGLLGTVT |
| | | | | 541 | EAEDRS |
| 28 | 30172039 | AAP20428 | Na+/H+ | 1 | MGLGVVAELV RLGVLSSTSD HASVVSINLF VALLCACIVL GHLLEENRWV NESTALIVGL |
| | | | antiporter | 61 | GTGTVILMIS RGVVIHVLVF SEDLFFFYLL PPIIFNAGFQ VKKKQFFRNF ITITLFGAVG |
| | | | NHX1 | 121 | TLISFTVISL GALGLISRLN IGALELGDYL ALGAIFSATD SVCTLQVLSQ DETPFLYSLV |
| | | | Zea mays | 181 | FGEGVVNDAT SVVVFNALQN FDITHIDAEV VFHLLGNFFY LFLLSTVLGV ATGLISALVI |
| | | | subsp. | 241 | KKLYFGRHST DREVALMMLM AYLSYMLAEL FALSGILTVF FGCIVMSHYT WHNVTESSRI |
| | | | mays | 301 | TTKHAFATLS FLAETFLFLY VGMDALDIDK WRSVSDTPGK SLAISSILMG LVMVGRAAFV |
| | | | | 361 | FPLSFLSNLA KKTEHEKISW KQQVVIWWAG LMRGAVSMAL AYKKFTRAGH TQVRGNAIMI |
| | | | | 421 | TSTIIVVLFS TMVFGLLTKP LINLLIPHRN ATSMLSDDSS PKSLHSPLLT SQLGSDLEEP |
| | | | | 481 | TNIPRPSSIR GEFLTMTRTV HRYWRKFDDA FMRPMFGGRG FVPFVPGSPT ERNPPDLSKA |
| 29 | 30172041 | AAP20429 | Na+/H+ | 1 | MGLGVDAETV RLGVLSSTSD HASVVSNNFF VALLCACIVL CHLLEENRMV NESITALLVG |
| | | | antiporter | 61 | LGTGTVILMI SRGVSIHVLV FSEDLFFIYL LPPIIFWAGF QVKKKQFFRN FITIILFGAI |
| | | | NHX2 | 121 | GTLISFVIIS LGAMGLFKKL DVGPLELGDY LAIGAIFSAT DSVCTLQVLN QDETPLLYSL |
| | | | Zea mays | 181 | VFGEGVVNDA TSIVVFNALQ NFDITHINAE VVFHLLGNFL YLFLLSTVLG VATGLISALV |
| | | | subsp. | 241 | IKKIYFGRHS TDREVALMML MAYLSYMLAE LFALSGILTV FFGCIVMSHY TWHNVTESSR |
| | | | mays | 301 | ITTKHAFATL SFLAETFIFL YVGMDALDIE KWRSVSDTPG KSIAISSILM GLVMLGRAAF |
| | | | | 361 | VFPLSFLSNL AKKNEHEKIS WKQQVVIWWS GLMRGAVSMA LAYNKFTRAG HTEVRGNEIM |
| | | | | 421 | ITSTITVVLF STVVFGLLTK PLIRLLMPHR HLTMLSDDST PKSLHSPLLT SQLGSSIEEP |
| | | | | 481 | TQIPRPTNIR GEFTTMTRTV HRYWRKFDDK FMRPMFGGRG FVPFVPGSPT ERNPHDLSKP |
| 30 | 32396168 | AAP20430 | Na+/H+ | 1 | MSIGLTAETV TNKLASAEHP QVVPNSVFIA LLCLCLVIGH LLEENRWVNE SITAILVGAA |
| | | | antiporter | 61 | TGTVILLISK GKSSHILVFD EELFFIYLLP PIIFNAGFQV KKKQFFRNFI TIILFGAICT |
| | | | NHX3 | 121 | LISFVIISLG AMGLFKKLDV GPLELGDYLA IGAIFSATDS VCTLQVLNQD ETPLLYSLVF |
| | | | Zea mays | 181 | GEGVVNDATS VVLFNAVQKI DFEHLTGEVA LQVFGNFLYL FSTSTVLGIA TGLITAFVLK |
| | | | subsp. | 241 | TLYFGRHSTT RELAIMVLMA YLSFMLAELF SLSGIITVFF CGVLMSHVTW HNVTESSRIT |
| | | | mays | 301 | SRBVFAMLSF IAETFLFLYV GTDALDFTKW KTSSLSFGKS LGVSSVLLGL VLVGRAAFVF |
| | | | | 361 | PLSFLSNLSK KHPGEKITIR QQVVIWWAGL MRGAVSIALA FNKFTRAGHT QVRGNAIMIT |
| | | | | 421 | STIIVVLFST VVFGLLTKPL INLLIPHRNA TSMLSDDSSP KSLHSPLLTS QLISSIEEPT |
| | | | | 481 | QIPRPTNIRG EFMTMTRTVH RYWRKFDDKF MRPMFGGRGF VPFVPGSPTE RSSPDLSKA |
| 31 | 32396170 | AAP20431 | Na+/H+ | 1 | MGYQVVAAQL KLASSADHAS VVIITLFVAL LCACIVLGHL LEENRWLNES ITALIIGLGT |
| | | | antiporter | 61 | GVVILLISRG KNSRLLVFSE DLFFIYLLPP IIFNAGFQVK KKQFFRNFMT ITLFGAVGTM |
| | | | NHX4 | 121 | ISFFTISLGA IATFSRMSIG TLDVGDFLAI GAIFSATDSV CTLQVLHQDE TPFLYSLVFG |
| | | | Zea mays | 181 | EGVVNDATSV VLFNAVQKIQ FTHINAWTAL QLIGNFLYLF STSTLLGIGT GLITAFVLKK |
| | | | subsp. | 241 | LYFGRHSTTR ELAIMILMAY LSYMLAELFS LSGLLTVFFC GVLMSHVTWH NVTESSRTTS |
| | | | mays | 301 | RHVFATLSFI SETFIFLYVG MDALDFEKWK TSSLSFGGTL GVSGVLMGLV MLGRAAFVFP |
| | | | | 361 | LSFLSNLAKK HQSEKISFRM QVVIWWAGLM RGAVSMALAL NKFTRSGHTQ LHGNAIMITS |
| | | | | 421 | TITVVLFSTM VFGMITKPLI RLLLPASGHP RELSEPSSPK SFHSPLLTSQ QGSDLESTTN |
| | | | | 481 | IVRPSSLRGL LTKPTHTVHY YWRKFDDALM RPVFGGRGFV PFVPGSPTER NPPDLSKA |
| 32 | 32396174 | AAP20432 | Na+/H+ | 1 | MSMGYQVVAA QLKVASSADH ASVVIITLFV ALLCACIVLG HLLEENRWLN ESITALIIGL |
| | | | antiporter | 61 | CTGGVILMTT KGKSSHVLVF SEDLFFIYLL PPIIFIAGFQ VKKKQFFRNF MTITLFGAVG |
| | | | NHX5 | 121 | TMISFFTISL GAIAIFSRMN IGTLDVGDFL AIGAIFSATD SVCTLQVLHQ DETPFLYSLV |
| | | | Zea mays | 181 | FGEGVVNDAT SVVLFNAVQK IQITHINAEV ALQVFGNFLY LFSTSTLLGI ATGLITSFVL |
| | | | subsp. | 242 | KKLYFARHST TRELAIMMLM AYLSYMLAEL FSLSGILTVF FCGVLMSHVT WHNVTESSRI |
| | | | mays | 301 | TSRHVFAMLS FIAETFIFLY VGTDALDFDK WKTSSLSFGG TLGVSALIMA LVLLGRAAFV |
| | | | | 361 | FPLSVLTNFS NKHENESITF KHQVIIWWAG LMRGAVSIAL AFKQFTYSGV TLDPVNAAMV |
| | | | | 421 | TNTTIVVLFT TLVFGLLTKP LIRLLMPHRH LTMLSDDSTP KSLHSPLLTS QLGSDLEEPT |
| | | | | 481 | NIPRPSSIRG EFLTMTRTVH RYWRKFDDAF MRPMFGGRGF VPVVPGSPIE RSVPQWSEEA |
| | | | | 541 | HNKEP |
| 33 | 32396176 | AAP20433 | Na+/H+ | 1 | MGLGVVAELV RLGVLSSTSD HASVVSINLF VALLCACIVL GHLLEENRWV NESITALIIG |
| | | | antiporter | 61 | LCTGVVILLT TKGKSSHILV FSEDLFFIYL LPPIIFNAGF QVKKKQFFRN FMTITLFGAV |
| | | | NHX6 | 121 | GTMISFPTIS LGALGLISRL WIGALELGDY LALGAIFSAT DSVCTLQVLS QDETPFLYSL |
| | | | Zea mays | 181 | VFGEGVVNDA TSVVVFNALQ NFDITNIDAE VVFNLLGMFF YLFLLSTVLG VATGLISALV |
| | | | subsp. | 241 | IKKLYFGRHS TDREVALMML MAYLSYMLAE LFALSGILTV FFGCIVMSHY TWHNVTESSR |
| | | | mays | 301 | ITTKHAFATL SFLAETFLFL YVGMDALDID KWRSVSDTPG KSLAISSILM GLVMVGRAAF |
| | | | | 361 | VFPLSFLSNL AKKTEHEKIS WKQQVVIWWA GLMRGAVSMA LAYKKFTRAG HTQVRGNAIM |
| | | | | 421 | ITSTIIVVLF STMVFGLLTK PLINLLIPHR NATSMLSDDS SPKSLHSPLL TSQLGSDLEE |
| | | | | 481 | PTNIPRFSSI RGEFLTMTRT VHRYWRKFDD AFMRPMFGGR GFVPFVPGSP TERNPPDLSK |
| | | | | 541 | A |
| 34 | 22902099 | AAM54141 | Na+/H+ | 1 | MVAPQLAAVF TKLQTLSTSD HASVVSMWIF VALLCACIVI GHLLEENRWM NESITALIIG |
| | | | antiporter | 61 | VFTGVIILLT SGGKSSHLLV FSEDLFFIYL LPPIIFNAGF QVKKKQFFRN PITIMLFGAV |
| | | | Gossypium | 121 | GTLISCTIIS LGVINFFKEM DIGSLDIGDF LAIGAIFAAT DSVCTLQVLN QDETPLLYSL |
| | | | hirsutum | 181 | VFGEGVVNDA TSVVLFNAIQ SFDLVNTSPR ILLEFIGSFL YLFLASTMLG VIVGLVSAYI |
| | | | | 241 | IKKLYFGRHS TDREFALMML MAYLSYIMAE LFYLSGILTV FFCGIVMSHY TWHNVTESSR |
| | | | | 301 | VTTKHAFATL SFVAETFLFL YVGMDALDME KWRFVSDSPG TSVAVSAVLM GLVMVGRAAF |
| | | | | 361 | VFPLSFLSNL AKKSTSEKIS FREQIIIWWA GLMRGAVSMA LAYNQFTRGG HTQLRGNAIM |
| | | | | 421 | ITSTITIVLF STVVFGLMTK PLIRFLLPHP KPTASMLSDQ STPKSMEAPF LGSGQDSFDD |
| | | | | 481 | SLIGVHRPNS IRALLTTPAH TVHYYWRKFD NAFMRPMFGG RGFVPFVPGS PTERSEPNLP |
| | | | | 541 | QWQ |
| 35 | 30144703 | AAP15178 | Na+/H+ | 1 | MWSQLSSFFA SKMDMVSTSD HASVVSMNLF VALLCGCIVI GHLLEENRWM NESITALLIG |
| | | | antiporter | 61 | LSTGIIILLI SGGKSSHLLV FSEDLFFIYL LPPIIFNAGF QVKKKQFFRN FITIILFGAV |
| | | | Suaeda | 121 | GTLVSFIIIS LGSIAIFQKM DIGSLELGDL LAIGAIFAAT DSVCTLQVLN QDETPLLYSL |
| | | | maritima | 181 | VFGEGVVNDA TSVVLFNAIQ NFDLTHIDHR IAYRIAFQFG GNFLYLFFAS TLLGAVTGLL |
| | | | subsp. | 241 | SAYVIKKLYF GRHSTDREVA LMMLMAYLSY MLAELFYLSG ILIVFFCGIV MSHYTWHNVT |
| | | | salsa | 301 | ESSRVTTKHA FATLSFVAEI FIFLYVGMDA LDIEKWRFVS DSPGTSVAVS SILLGLLMVG |
| | | | | 361 | RALLFSLVFL MNLSKKSNSE KVTFNQQIVI WWAGLMRGAV SVALAYNQFS RSGHTQLRGN |
| | | | | 421 | AIMITSTITV VLFSTMVFGL LTKPLILFML PQPKHFTSAS TVSDLGSPKS FSLPLLEDRQ |
| | | | | 481 | DSEADLGNDD EEAYPRGTIA RPTSLRMLLN APTHTVHHYW RRFDDYFMRP VFGGRGFVPF |
| | | | | 541 | VPGSPTEQST TNLSQRT |
| 36 | 28201131 | BAC56698 | Na+/H+ | 1 | MAFEVVAAQL ARLSDALATS DHASVVSINL FVALLCACIV LGHLLEENRW LNESITALII |
| | | | antiporter | 61 | GLCTGVVILM TTKGKSSHVL VFSEDLFFIY LLPPIIFNAG FQVKKKQFFR NFMTITLFGA |
| | | | Hordeum | 121 | VGTMISFFTI SLAAIAIFSK MNIGTLDVSD FLAIGAIFSA TDSVCTLQVL NQDETPFLYS |
| | | | vulgare | 181 | LVFGEGVVND ATSVVLFNAL QNFDPNQIDA IVILKFLGNF CYLFVSSTFL GVFSGLLSAY |
| | | | | 241 | IIKKLYIGRH STDREVALMM LMAYLSYMLA ELLDLSGILT VFFCGIVMSH YTWHNVTESS |
| | | | | 301 | RVTTKHAFAT LSFIAETFLF LYVGMDALDI EKWKFASDSP GKSIGISSIL LGLVLVGRAA |
| | | | | 361 | FVFPLSFLSN LTKKTELEKI SWRQQIVIWW AGLMRGAVSI ALAYNKFTRS GHTQLHGNAI |
| | | | | 421 | MTTSTITVVL FSTMLFGILT KPLIRFLLPA SSNGDPSEPS SPKSLHSPLL TSMLGSDMEA |
| | | | | 481 | PLPIVRPSSL RMLITKPTHT IHYYWRKFDD ALMRPMFGGR GFVPYSPGSP TDPNVIVA |
| 37 | 27948863 | AAO25547 | Na+/H+ | 1 | MGWGLGDPPA DYGSIMAVGL FVALMCICII VGHLLEENRW MNESTTALLL GLGAGTVILF |
| | | | antiporter | 61 | ASSGKNSRLM VFSEDLFFIY LLPPIIFNAG FQVKKKQFFR NFMTITLFAV VGTLISFSII |
| | | | Hordeum | 121 | SLGAMGLISR LNIGALELGD YLALGAIFSA TDSVCTLQVL SQDETPFLYS LVFGEGVVND |
| | | | bre- | 181 | ATSVVLFNAI QNFDLGNFSS LKFLQFIGNF LYLFGASTFL GVASGLLSAY VIKKLYFGRH |
| | | | visubulatum | 241 | STDREVAIMM LMAYLSYMLA ELLDLSGILT VFFCGIVMSH YTWHNVTESS RVTTKHAFAT |
| | | | | 301 | LSFISETFLF LYVGMDALDI EKWKIVSETY SPMKSITLSS IILALVLVAR AAFVFPLSYL |
| | | | | 361 | SNLTKKTAGE KISIRQQVII WWAGLMRGAV SIALAYNKFA KSGHTQLPSN AIMITSTIII |
| | | | | 421 | VLFSTIVFGL LTKPLIRLLI PARHLTREVS ALSEPSSPKS FLEQLTVNGP ETDVENGVSI |
| | | | | 481 | RRPTSLRMLL ASPTRSVHHY WRKFDNAFMR PVFGGRGFVP FVPGSPTESS VPLLAHGSEN |
| 38 | 29825705 | AAO91943 | Vacuolar | 1 | MGPDLGALAL RYTGLAVSDH DSIVAINIFI ALLCGCIVFG HLLEGNRWVN ESTTAIVLGL |
| | | | Na+/H+ | 61 | ITGGVILLCT KGVNSRILIF SEDIFFIYLL PPIIFNAGFQ VKKKQFFRNF ATIILFGAVG |
| | | | antiporter | 121 | TLISFVIITL GAMGLFRKLD VGPLELGDYL AIGAIFSATD SVCTLQVLNQ DQAPLLYSLV |
| | | | Hordeum | 181 | FGEGVVNDAT SVVLFNAIQN IDLNHFDVLV LLQLICKFLY LFLTSTVLGV AAGLLSAYII |
| | | | vulgare | 241 | KKLCFARHST DREVAIMILM AYLSYMLSML LDLSGILTVF FCGIVMSHYT RHNVTESSRV |
| | | | | 301 | TTKHTFATLS FIAEIFLFLY VGMDALDIDK WKLASSSPKK PIALSAVILG LVMVGRAAFV |
| | | | | 361 | FPLSYLSNLS KKESHPKISF NQQVIIWWAG LMRGAVSIAL AYNKYTTSGH TAVRVNAVMI |
| | | | | 421 | TSTIIVVLFS TMVFGLLTKP LINLLVPPRP GTAADISSQS FLDPLTASLL GSDFDVGQLT |
| | | | | 481 | PQTNLQYLLT MPSRSVHRVW RKFDDKFMRP MFGGRGFVPF VPGSPIERSV HGPGLLGTVT |
| | | | | 541 | EAENRS |

TABLE IV
Relative yield decrease of representative plants.

| | RELATIVE YIELD DECREASE | | | |
|---|---|---|---|---|
| | 25% | | 50% | |
| CROP | (mmho/cm) | (mM NaCl) | (mmho/cm) | (mM NaCl) |
| Barley | 13 | 120 | 18 | 170 |
| Sugarbeet | 11 | 105 | 15 | 150 |
| Sorghum | 7.2 | 65 | 11 | 100 |
| Soybean | 6.2 | 59 | 7.5 | 65 |
| Rice | 3.8 | 36 | 5.9 | 50 |
| Corn | 3.8 | 36 | 5.9 | 50 |
| Alfalfa | 5.4 | 45 | 8.8 | 75 |
| Cucumber | 4.4 | 40 | 7.0 | 65 |
| Potato | 2.8 | 36 | 5.9 | 50 |
| Beans | 2.3 | 18 | 3.2 | 28 |
| Grape | 4.1 | 37 | 6.7 | 62 |
| Orange | 3.2 | 28 | 4.8 | 43 |
| Peach | 2.9 | 25 | 4.1 | 35 |
| Strawberry | 1.8 | 14 | 2.5 | 21 |

1. A non-naturally occurring non-halophyte oil crop plant comprising seeds with normal or near normal fatty acid distribution when cultivated in high salt.

2. The non-naturally occurring non-halophyte oil crop plant of claim 1 wherein the near normal fatty acid distribution is within five percent of the normal distribution.

3. The non-naturally occurring non-halophyte oil crop plant of claim 1 wherein the high salt is at least two times the optimal osmolality for the non-halophyte plant.

4. The non-naturally occurring non-halophyte oil crop plant of claim 1 wherein the high salt is at least three times the optimal osmolality for the non-halophyte plant.

5. The non-naturally occurring non-halophyte oil crop plant of claim 1 wherein the high salt is at least four times the optimal osmolality for the non-halophyte plant.

6. The non-naturally occurring non-halophyte oil crop plant of claim 1 wherein the high salt is at least five times the optimal osmolality for the non-halophyte plant.

7. The non-naturally occurring non-halophyte oil crop plant of claim 1 wherein the high salt is at least ten times the optimal osmolality for the non-halophyte plant.

8. The non-naturally occurring non-halophyte oil crop plant of claim 1 wherein the high salt is at least twenty times the optimal osmolality for the non-halophyte plant.

9. The non-naturally occurring non-halophyte oil crop plant of claim 1 wherein the plant is selected from the group consisting of canola, safflower, palm, coconut, cotton, flax, olive, jojoba, peanut, castor, sesame, sunflower and soybean.

10. The non-naturally occurring non-halophyte oil crop plant of claim 1 wherein the plant is canola.

11. The non-naturally occurring non-halophyte oil crop plant of claim 1 wherein the plant is safflower.

12. The non-naturally occurring non-halophyte oil crop plant of claim 1 wherein the plant comprises a transgene.

13. The non-naturally occurring non-halophyte oil crop plant of claim 12 wherein the transgene induces vacuolar accumulation of salt.

14. The non-naturally occurring non-halophyte oil crop plant of claim 12 wherein the transgene induces secretion of salt out of the cytoplasm.

15. The non-naturally occurring non-halophyte oil crop plant of claim 12 wherein the transgene induces exclusion of salt from the cytoplasm.

16. The non-naturally occurring non-halophyte oil crop plant of claim 13 wherein the transgene comprises a first nucleic acid encoding a vacuolar Na+/H+ transporter.

17. The non-naturally occurring non-halophyte oil crop plant of claim 16 wherein the first nucleic acid is selected from the group consisting of the following:
(a) a nucleic acid molecule of the coding strand shown in SEQ ID NO:1, or a complement thereof;
(b) a nucleic acid molecule encoding the same amino acid sequence as encoded by the nucleotide sequence of (a);
(c) a nucleic acid molecule that hybridizes to the sequence set forth in SEQ ID NO:1 or the complement of the sequence set forth in SEQ ID NO:1 under highly stringent conditions that include at least one wash in 0.1×SSC, 0.1% SDS, at 65° C. for thirty minutes; and
(d) a nucleic acid molecule encoding a plant NHX transporter polypeptide that hybridizes to the sequence set forth in SEQ ID NO:1 or the complement of the sequence set forth in SEQ ID NO:1 under moderately stringent conditions that includes at least one wash in 0.1×SSC, 0.1% SDS, at 50° C. for thirty minutes.

18. The non-naturally occurring non-halophyte oil crop plant of claim 17 wherein the transgene further comprises a second nucleic acid operably linked to the first nucleic acid, where in the second nucleic acid comprises a plant promoter.

19. The non-naturally occurring non-halophyte oil crop plant of claim 18 wherein the promoter is the CaMV promoter.

20. The non-naturally occurring non-halophyte oil crop plant of claim 19 wherein the plant is canola.

21. A transgenic non-halophyte seed produced from the plant of claim 1.
